(19) United States
(12) Patent Application Publication
(10) Pub. No.: US 2004/0220411 A1
(43) Pub. Date: Nov. 4, 2004

(54) Oxalic acid derivatives

(76) Inventors: Werner Mederski, Zwingenberg (DE); Bertram Cezanne, Morfelden-Walldorf (DE); Dieter Dorsch, Ober-Ramstadt (DE); Christos Tsaklakidis, Weinheim (DE); Johannes Gleitz, Darmstadt (DE); Christopher Barnes, Soden (DE)

Correspondence Address: Millen White, Zelano & Branigan, Arlington Courthouse Plaza I, 2200 Clarendon Boulevard Suite 1400, Arlington, VA, 22201, US

(21) Appl. No.: 10/474,969

(22) PCT Filed: Mar. 18, 2002
(86) PCT No.: PCT/EP02/02963

(30) Foreign Application Priority Data
Apr. 10, 2001 (DE) 101 17 823.9

(51) Int. Cl. C07D 271/12

(52) U.S. Cl. 548/131; 548/132; 562/450; 564/86; 558/392; 564/153

(57) ABSTRACT

Novel compounds of the formula (I) in which R¹, R², R³, R⁴, X and Z are as defined in Patent claim 1, are inhibitors of coagulation factor Xa and can be employed for the prophylaxis and/or therapy of thromboembolic disorders and for the treatment of tumours.

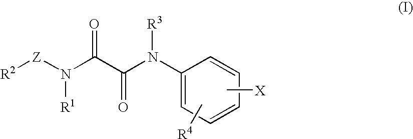

The invention relates to compounds of the formula I

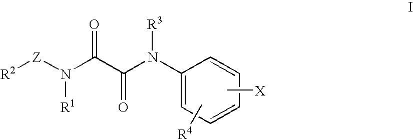

in which

R¹ and R³, independently of one another, are H or A, Ar, Ar-alk, Het, Het-alk or acyl,

R² is Ar or Het,

R⁴ is H, A, OH, OA′, OAr, Ar-alk-O, O-acyl, COOH, COOA′, CONH₂, CONHA′, CONA′₂, CN, NHA′, NA′₂, NHCH₂Ar′, NH-acyl or Hal,

X is Ar, Ar-alk or U,

Ar is phenyl, naphthyl or biphenyl, each of which is unsubstituted or monosubstituted or polysubstituted by A′, Hal, OH, OA′, OCH₂Ar′, O-acyl, COOH, COOA′, CONH₂, CONHA′, CONA′₂, CONHNH₂, CH₂NH₂, CH₂NHA′, CH₂NA′₂, CH₂CH₂NH₂, CH₂NH-acyl, CN, NH₂, NHA′, NA′₂, NHCH₂Ar′, NHCOAr′, C(═NH)NH₂, C(═NH)NH-COOA′, SO₂CH₂R⁶, SO₂NR⁸R⁹,

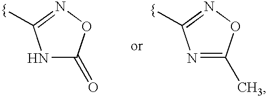

Ar′ is phenyl, naphthyl or biphenyl, each of which is unsubstituted or monosubstituted or polysubstituted by A′, Hal, OH, OA′, O-acyl, COOH, COOA′, CONH₂, CONHA′, CONA′₂, CONHNH₂, CH₂NH₂, CH₂NHA′, CH₂NA′₂, CH₂CH₂NH₂, CH₂NH-acyl, CN, NH₂, NHA′, NA′₂, C(═NH)NH₂, SO₂CH₂R⁶ or SO₂NR⁸R⁹,

Het is a monocyclic or bicyclic aromatic heterocyclic radical having from 1 to 4 N, O and/or S atoms which is unsubstituted or monosubstituted or polysubstituted by A′, Hal, OH, OA′, OCH₂Ar′, O-acyl, COOH, COOA′, CONH₂, CONHA′, CONA′₂, CONHNH₂, CH₂NH₂, CH₂NHA′, CH₂NA′₂, CH₂CH₂NH₂, CH₂NH-acyl, CN, NHA′, NA′₂, NHCH₂Ar′, NHCOAr′, C(═NH)NH₂, SO₂CH₂R⁶, SO₂NR⁸R⁹,

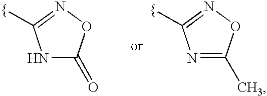

U is a radical of the formula IIa, IIb, IIc or IId 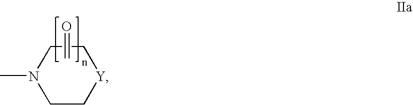 (CH₂)ₚ—SO₂—(CH₂)ₙ—R⁶ IIb,

(CH₂)ₚ—SO₂—NR⁸R⁹ IIc,

(CH₂)ₚ—NH—SO₂—(CH₂)ₙ—R⁶ IId,

which is unsubstituted or monosubstituted or polysubstituted by A′, Hal, OH, OA′, OCH₂Ar′, O-acyl, COOH, COOA′, CONH₂, CONHA′, CONA′₂, CONHNH₂, CH₂NH₂, CH₂NHA′, CH₂NA′₂, CH₂CH₂NH₂, CH₂NH-acyl, CN, NHA′, NA′₂, NHCH₂Ar′, NHCOAr′, C(═NH)NH₂, SO₂CH₂R⁶, SO₂NR⁸R⁹,

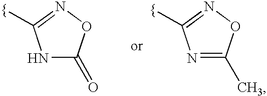

Y is O, S, NR⁵ or an alkylene chain (CH₂)ₘ, which is unsubstituted or monosubstituted or polysubstituted by OH, OA′, OAr′, O-acyl, COOH, COOA′, CONH₂, CONHA′, CONA′₂, CN, NH₂, NHA′, NA′₂, NHCH₂Ar′, NH-acyl, NHCOAr′, C(═NH)NH₂ or Hal and which may be interrupted by O, S or NR⁵,

Z is O, NR⁵ or an alkylene chain (CH₂)ₘ, which is unsubstituted or monosubstituted or polysubstituted by OH, OA′, OAr′, O-acyl, COOH, COOA′, CONH₂, CONHA′, CONA′₂, CN, NH₂, NHA′, NA′₂, NHCH₂Ar′, NH-acyl, NHCOAr′, C(═NH)NH₂ or Hal,

A is unbranched or branched alkyl having 1-8 carbon atoms, which is unsubstituted or monosubstituted or polysubstituted by OH, OA′, OAr′, O-acyl, COOH, COOA′, CONH₂, CONHA′, CONA′₂, CN, NH₂, NHA′, NA′₂, NHCH₂Ar′, NH-acyl, NHCOAr′, C(═NH)NH₂ or Hal and in which one or two CH₂ groups may be replaced by O or S atoms and/or by —CH═CH— groups and/or, in addition, 1-7 H atoms may be replaced by F,

A′ is unbranched or branched alkyl having 1-8 carbon atoms,

R⁵ is H, A, Ar, Ar-alk, Het, CO-T-R⁶ or SO₂-T-R⁶, and, if Y=NR⁵, R⁵ may alternatively be —C(═NH)—R⁷,

T is absent or is an alkylene chain having 1-5 carbon atoms, alkenylene chain having 2-5 carbon atoms or alkynylene chain having 2-5 carbon atoms, each of which is unsubstituted or monosubstituted or polysubstituted by OH, OA′, OAr′, O-acyl, COOH, COOA′, CONH₂, CONHA′, CONA′₂, CN, NH₂, NHA′, NA′₂, NHCH₂Ar′, NH-acyl, NHCOAr′, C(═NH)NH₂ or Hal,

R⁶ is H, A, Ar, Ar-alk or Het,

R⁷ is H, A′, Ar-alk or NR⁸R⁹,

R⁸ and R⁹, independently of one another, are H, A, Ar, Ar-alk, Het, acyl, Q¹ or Q², or, together with the nitrogen to which they are bonded, are a monocyclic saturated, unsaturated or aromatic heterocyclic radical having from 1 to 3 N, O and/or S atoms, which is unsubstituted or monosubstituted or polysubstituted by A′, Hal, OH, OA′, OCH₂Ar′, O-acyl, COOH, COOA′, CONH₂, CONHA′, CONA′₂, CONHNH₂, CH₂NH₂, CH₂NHA′, CH₂NA′₂, CH₂CH₂NH₂, CH₂NH-acyl, CN, NHA′, NA′₂, NHCH₂Ar′, NHCOAr′, C(═NH)NH₂ or SO₂CH₂R⁶,

Q¹ is a cycloalkyl radical, which is unsubstituted or monosubstituted or disubstituted by A′,

Q² is a monocyclic saturated or unsaturated heterocyclic radical having from 1 to 3 N, O and/or S atoms, which is unsubstituted or monosubstituted or disubstituted by A′, Hal, OH, OA′, OCH₂Ar′, O-acyl, COOH, COOA′, CONH₂, CONHA′, CONA′₂, CONHNH₂, CH₂NH₂, CH₂NHA′, CH₂NA′₂, CH₂CH₂NH₂, CH₂NH-acyl, CN, NHA′, NA′₂, NHCH₂Ar′, NHCOAr′, C(═NH)NH₂ or SO₂CH₂R⁶,

Hal is F, Cl, Br or I,

alk is alkylene having 1, 2, 3, 4, 5 or 6 carbon atoms,

m is 0, 1, 2, 3 or 4,

n is 1, 2 or 3,

p is 1, 2, 3, 4 or 5,

and pharmaceutically tolerated salts, solvates and stereoisomers thereof.

The invention had the object of finding novel compounds having valuable properties, in particular those which can be used for the preparation of medicaments.

It has been found that the compounds of the formula I and their salts have very valuable pharmacological properties and are well tolerated. In particular, they exhibit factor Xa-inhibiting properties and can therefore be employed for combating and preventing thromboembolic disorders, such as thrombosis, myocardial infarction, arteriosclerosis, inflammation, apoplexia, angina pectoris, restenosis after angioplasty and claudicatio intermittens.

The compounds of the formula I according to the invention may furthermore be inhibitors of the coagulation factors factor VIIa, factor IXa and thrombin in the blood coagulation cascade.

Aromatic amidine derivatives having an antithrombotic action are disclosed, for example, in EP 0 540 051 B1, WO 98/28269, WO 00/71508, WO 00/71511, WO 00/71493, WO 00/71507, WO 00/171509, WO 00/71512, WO 00/71515 and WO 00/71516. cyclic guanidines for the treatment of thromboembolic disorders are described, for example, in WO 97/08165. Aromatic heterocyclic compounds having a factor Xa inhibitory activity are disclosed, for example, in WO 96/10022. Substituted N-[(aminoiminomethyl)phenylalkyl]azaheterocyclylamides as factor Xa inhibitors are described in WO 96/40679.

The antithrombotic and anticoagulant effect of the compounds according to the invention is attributed to the inhibitory action against activated coagulation protease, known by the name factor Xa, or to the inhibition of other activated serine proteases, such as factor VIIa, factor IXa or thrombin.

Factor Xa is one of the proteases involved in the complex process of blood coagulation. Factor Xa catalyses the conversion of prothrombin into thrombin. Thrombin cleaves fibrinogen into fibrin monomers, which, after crosslinking, make an elementary contribution to thrombus formation. Activation of thrombin may result in the occurrence of thromboembolic disorders. However, inhibition of thrombin may inhibit the fibrin formation involved in thrombus formation. The inhibition of thrombin can be measured, for example by the method of G. F. Cousins et al. in Circulation 1996, 94, 1705-1712.

Inhibition of factor Xa can thus prevent the formation of thrombin.

The compounds of the formula I according to the invention and their salts engage in the blood coagulation process by inhibiting factor Xa and thus inhibit the formation of thrombuses.

The inhibition of factor Xa by the compounds according to the invention and the measurement of the anticoagulant and antithrombotic activity can be determined by conventional in-vitro or in-vivo methods. A suitable method is described, for example, by J. Hauptmann et al. in Thrombosis and Haemostasis 1990, 63, 220-223.

The inhibition of factor Xa can be measured, for example by the method of T. Hara et al. in Thromb. Haemostas. 1994, 71, 314-319.

Coagulation factor VIIa initiates the extrinsic part of the coagulation cascade after binding to tissue factor and contributes to the activation of factor X to give factor Xa. Inhibition of factor VIIa thus prevents the formation of factor Xa and thus subsequent thrombin formation. The inhibition of factor VIIa by the compounds according to the invention and the measurement of the anticoagulant and antithrombotic activity can be determined by conventional in-vitro or in-vivo methods. A conventional method for the measurement of the inhibition of factor VIIa is described, for example, by H. F. Ronning et al. in Thrombosis Research 1996, 84, 73-81.

Coagulation factor IXa is generated in the intrinsic coagulation cascade and is likewise involved in the activation of factor X to give factor Xa. Inhibition of factor IXa can therefore prevent the formation of factor Xa in a different way. The inhibition of factor IXa by the compounds according to the invention and the measurement of the anticoagulant and antithrombotic activity can be determined by conventional in-vitro or in-vivo methods. A suitable method is described, for example, by J. Chang et al. in Journal of Biological Chemistry 1998, 273, 12089-12094.

The compounds according to the invention may furthermore be used for the treatment of tumours, tumour illnesses and/or tumour metastases. A correlation between tissue factor TF/factor VIIa and the development of various types of cancer has been indicated by T. Taniguchi and N. R. Lemoine in Biomed. Health Res. (2000), 41 (Molecular Pathogenesis of Pancreatic Cancer), 57-59.

The compounds of the formula I can be employed as medicament active ingredients in human and veterinary medicine, in particular for the treatment and prevention of thromboembolic disorders, such as thrombosis, myocardial infarction, arteriosclerosis, inflammation, apoplexia, angina pectoris, restenosis after angioplasty, claudicatio intermittens, venous thrombosis, pulmonary embolism, arterial thrombosis, myocardial ischaemia, unstable angina and strokes based on thrombosis. The compounds according to the invention are also employed for the treatment or prophylaxis of atherosclerotic diseases, such as coronary arterial disease, cerebral arterial disease or peripheral arterial disease. The compounds are also employed in combination with other thrombolytic agents in myocardial infarction, furthermore for prophylaxis for reocclusion after thrombolysis, percutaneous transluminal angioplasty (PTCA) and coronary bypass operations. The compounds according to the invention are furthermore used for the prevention of rethrombosis in microsurgery, furthermore as anticoagulants in connection with artificial organs or in haemodialysis. The compounds are furthermore used in the cleaning of catheters and medical aids in patients in vivo, or as anticoagulants for the preservation of blood, plasma and other blood products in vitro. The compounds according to the invention are furthermore used for diseases in which blood coagulation makes a crucial contribution toward the course of the disease or represents a source of secondary pathology, such as, for example, in cancer, including metastasis, inflammatory disorders, including arthritis, and diabetes.

The compounds according to the invention are furthermore used for the treatment of migraine (F. Morales-Asin et al., Headache, 40, 2000, 45-47).

In the treatment of the disorders described, the compounds according to the invention are also used in combination with other thrombolytically active compounds, such as, for example, with the “tissue plasminogen activator” t-PA, modified t-PA, streptokinase or urokinase The compounds according to the invention are administered either at the same time as or before or after the other substances mentioned. Particular preference is given to simultaneous administration with aspirin in order to prevent recurrence of the clot formation. The compounds according to the invention are also used in combination with blood platelet glycoprotein receptor (IIb/IIIa) antagonists, which inhibit blood platelet aggregation.

The invention relates to the compounds of the formula I and their salts and to a process for the preparation of compounds of the formula I according to Claim 1 and their salts, characterised in that

a) they are liberated from one of their functional derivatives by treatment with a solvolysing or hydrogenolysing agent by

i) liberating an amidino group from the hydroxyl, oxadiazole or oxazolidinone derivative by hydrogenolysis or solvolysis,

ii) replacing a conventional amino-protecting group with hydrogen by treatment with a solvolysing or hydrogenolysing agent, or liberating an amino group protected by a conventional protecting group,

or

b) a cyano group is converted into an N-hydroxyamidino group,

or

c) a compound of the formula II

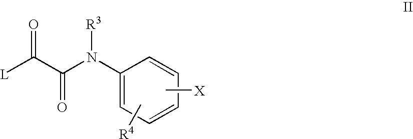

in which

L is Cl, Br, I or a free or reactively functionally modified OH group, and

R³, R⁴ and X are as defined in Claim 1, with the proviso that any free amino and/or hydroxyl group present is protected,

is reacted with a compound of the formula III

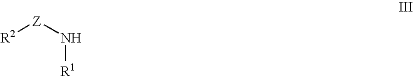

in which

R¹, R² and Z are as defined in Claim 1,

and, where appropriate, a protecting group is subsequently removed

or

d) a compound of the formula IV

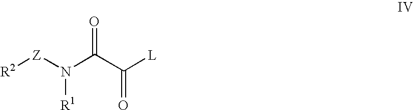

in which

L is Cl, Br, I or a free or reactively functionally modified OH group, and

R¹, R² and Z are as defined in Claim 1, with the proviso that any free amino and/or hydroxyl group present is protected,

is reacted with a compound of the formula V

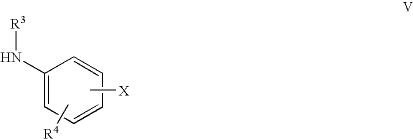

in which

R³, R⁴ and X are as defined in claim 1,

and, where appropriate, a protecting group is subsequently removed,

and/or

e) a base or acid of the formula I is converted into one of its salts.

The invention also relates to the optically active forms, the racemates, the diastereomers, and the hydrates and solvates, for example alcoholates, of these compounds.

The invention also relates to the prodrug compounds, i.e. derivatives of the compounds of the formula I which are readily converted into the actual active ingredients, such as, for example, esters or acylated amino compounds.

The invention also relates, in particular, to the —C(═NH)—NH₂ compounds of the formula I which are substituted by —COA, —COOA, —OH or by a conventional amino-protecting group.

For all radicals which occur more than once, such as, for example, A, their meanings are independent of one another. Above and below, the radicals and parameters R¹, R², R³, R⁴, X and Z are as defined under the formula I, unless expressly stated otherwise.

Alkyl, is unbranched (linear) or branched, and has 1, 2, 3, 4, 5, 6, 7 or 8 carbon atoms. Alkyl is preferably methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl. Alkyl is very particularly preferably alkyl having 1, 2, 3, 4, 5 or 6 carbon atoms, preferably methyl, ethyl, propyl, isopropyl, butyl, isobutyl, sec-butyl, tert-butyl, pentyl, hexyl, trifluoromethyl, pentafluoroethyl or 1,1,1-trifluoro- or 1,1,1-trichloroethyl, furthermore also, for example, 1-propenyl.

A is particularly preferably methyl, ethyl, propyl, isopropyl, butyl, isobutyl, tert-butyl, trifluoromethyl, 1,1,1-trifluoro- or 1,1,1-trichloroethyl, furthermore also, for example, 1-propenyl.

A′ is particularly preferably methyl, ethyl, propyl, isopropyl, butyl or isobutyl.

Cycloalkyl is preferably cyclopropyl, cyclobutyl, cyclopentyl, cyclohexyl, cycloheptyl, cyclopentenyl or cyclohexenyl.

Alkylene is preferably methylene, ethylene, propylene, butylene, pentylene or hexylene, furthermore branched alkylene.

“alk” is particularly preferably methylene or ethylene.

Alkenylene is preferably ethenylene, propenylene, butenylene, butadienylene, isobutenylene, pentenylene or pentadienylene.

Alkynylene is preferably acetylene, propynylene, butynylene, butadiynylene, pentynylene or pentadiynylene.

Acyl is preferably formyl, acetyl, propionyl, furthermore also butyryl, pentanoyl, hexanoyl or, for example, also benzoyl or SO₂A, where A is, in particular, methyl.

Ph is phenyl, Me is methyl, Et is ethyl, BOC is tert-butoxycarbonyl.

Hal is preferably F, Cl or Br, but also I.

Poly means di, tri, tetra or penta, preferably di or tri, particularly preferably di.

R¹is preferably H, A or Ar-alk, in particular, for example, H, alkyl having 1, 2, 3, 4, 5 or 6 carbon atoms, in which 1-5 H atoms may be replaced by F, or benzyl.

R² is preferably Ar, in particular phenyl which is monosubstituted or disubstituted by Hal, OH, OA′, COOH, COOA′, CONH₂, CONHNH₂, CH₂NH₂, CH₂NHA′, CH(NH₂)CH₂NH₂, C(═NH)NH₂, C(═NH)NH—-COOA′, SO₂NR⁸R⁹,

or

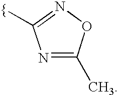

Z is preferably, for example, an unsubstituted alkylene chain (CH₂)ₘ, where m=0 or 1.

R³ is preferably H or A, particularly preferably H or alkyl having 1, 2, 3, 4, 5 or 6 carbon atoms, in which 1-5 H atoms may be replaced by F or chlorine.

R⁴ is preferably H, F or Cl.

X is preferably, for example, phenyl which is monosubstituted or disubstituted by CH₂CH₂NH₂, C(═NH)NH₂, SO₂CH₂R⁶ or SO₂NR⁸R⁹, or an unsubstituted radical of the formula IIa, IIb, IIc or IId 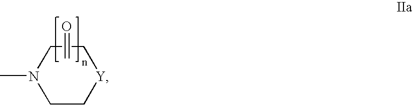 (CH₂)ₚ—SO₂—(CH₂)ₙ—R⁶ IIb,

(CH₂)ₚ—SO₂—NR⁸R⁹ IIc,

(CH₂)ₚ—NH—SO₂—(CH₂)ₙ—R⁶ IId.

X is particularly preferably, for example, phenyl which is monosubstituted or disubstituted by CH₂CH₂NH₂, C(═NH)NH₂, SO₂CH₂R⁶ or SO₂NR⁸R⁹, or an unsubstituted radical of the formula IIa or IId 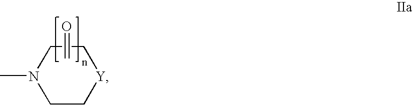 (CH₂)ₚ—NH—SO₂—(CH₂)ₙ—R⁶ IId.

X is very particularly preferably, for example, phenyl which is monosubstituted or disubstituted by CH₂CH₂NH₂, C(═NH)NH₂, SO₂A″ or SO₂NH₂, or an unsubstituted radical of the formula IIa or IId 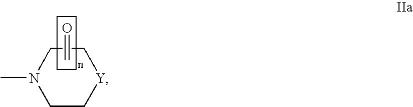 (CH₂)ₚ—NH—SO₂—CH₃ IId,

where

A″ is alkyl having 1, 2, 3, 4, 5 or 6 carbon atoms,

n is 1 or 2,

p is 1 or 2,

Y is O, NR⁵ or an unsubstituted alkylene chain (CH₂)_{m′},

m′ is 0, 1 or 2.

R⁵ is preferably H.

T is preferably not present (absent).

R⁶ is preferably H or A, in particular H or alkyl having 1, 2, 3, 4, 5 or 6 carbon atoms. R⁶ is very particularly preferably H.

R⁷ is preferably NH₂.

R⁸ is preferably H.

R⁹ is preferably H, A, benzyl, Het, Q¹ or Q².

R⁸ and R⁹, together with the nitrogen to which they are bonded, are preferably, for example, pyrrolidinyl, piperidinyl, piperazinyl, morpholinyl, tetrahydropyrimidinyl, dihydropyridinyl or dihydroimidazolyl, very particularly preferably piperidinyl or tetrahydropyrimidinyl.

Ar is preferably, for example, phenyl, further preferably monosubstituted o-, m- or p-tolyl, o-, m- or p-ethylphenyl, o-, m- or p-isopropylphenyl, o-, m- or p-fluorophenyl, o-, m- or p-chlorophenyl, o-, m- or p-hydroxyphenyl, o-, m- or p-methoxyphenyl, o-, m- or p-ethoxyphenyl, o-, m- or p-benzyloxyphenyl, o-, m- or p-acetoxyphenyl, o-, m- or p-propionyloxyphenyl, o-, m- or p-carboxyphenyl, o-, m- or p-methoxycarbonylphenyl, o-, m- or p-ethoxycarbonylphenyl, o-, m- or p-aminocarbonylphenyl, o-, m- or p-(N-methylaminocarbonyl)phenyl, o-, m- or p-(N,N-dimethylaminocarbonyl)phenyl, o-, m- or p-hydrazinocarbonylphenyl, o-, m- or p-aminomethylphenyl, o-, m- or p-(N-methylaminomethyl)phenyl, o-, m- or p-(N,N-dimethylaminomethyl)phenyl, o-, m- or p-aminoethylphenyl, o-, m- or p-formylaminomethylphenyl, o-, m- or p-acetamidomethylphenyl, o-, m- or p-cyanophenyl, o-, m- or p-aminophenyl, o-, m- or p-(N-methylamino)phenyl, o-, m- or p-(N,N-dimethylamino)phenyl, o-, m- or p-(N-benzylamino)phenyl, o-, m- or p-(N-benzoylamino)phenyl, o-, m- or p-amidinophenyl, o-, m- or p-(N-methoxycarbonylamidino)phenyl, o-, m- or p-methylsulfonylphenyl, o-, m- or p-aminosulfonylphenyl, or phenyl which is substituted in the o-, m- or p-position by

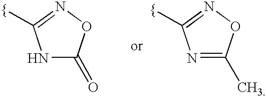

Ar is particularly preferably phenyl which is monosubstituted by Hal, OH, OA′, COOH, COOA′, CONH₂, CONHNH₂, CH₂NH₂, CH₂CH₂NH₂, CH₂NHA′, CH(NH₂)CH₂NH₂, C(═NH)NH₂, C(═NH)NH—COOA′, SO₂A′, SO₂NR⁸R⁹,

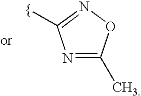

Ar′ is preferably phenyl.

U is preferably an unsubstituted radical of the formula IIa or IId 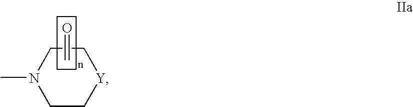 (CH₂)ₚ—NH—SO₂—(CH₂)ₙ—R⁶ IId.

U is very particularly preferably an unsubstituted radical of the formula IIa or IId 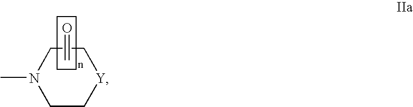 (CH₂)ₚ—NH—SO₂—CH₃ IId,

where

n is 1 or 2,

p is 1 or 2,

Y is O, NR⁵ or an unsubstituted alkylene chain (CH₂)ₘ,

R⁵ is H,

m is 0, 1 or 2.

The unsubstituted or substituted monocyclic or bicyclic aromatic heterocyclic radical having from 1 to 4 N, O and/or S atoms in Het is, for example, 2- or 3-furyl, 2- or 3-thienyl, 1-, 2- or 3-pyrrolyl, 1-, 2-, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1 ,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, further preferably 1,3-benzodioxol-5-yl, 1,4-benzodioxan-6-yl, 2,1,3-benzothiadiazol-4- or -5-yl or 2,1,3-benzoxadiazol-5-yl. Het is preferably unsubstituted.

The unsubstituted or substituted monocyclic saturated or unsaturated heterocyclic radical in Q² is preferably, for example, 2,3-dihydro-2-, -3-, -4- or -5-furyl, 2,5-dihydro-2-, -3-, -4- or -5-furyl, tetrahydro-2- or -3-furyl, 1,3-dioxolan-4-yl, tetrahydro-2- or -3-thienyl, 2,3-dihydro-1-, -2-, -3-, -4- or -5- pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl or 2,3-dihydro-2-oxofuranyl.

Q² is particularly preferably pyrrolidinyl, piperidinyl, piperazinyl, morpholinyl, tetrahydropyrimidinyl, dihydropyridinyl or dihydroimidazolyl, very particularly preferably piperidinyl or tetrahydropyrimidinyl.

The radical of the formula IIa is particularly preferably a monocyclic saturated heterocyclic radical having 1 or 2 N or O atoms which is monosubstituted or disubstituted by carbonyl oxygen, such as, for example, morpholin4-yl, 2-oxopiperidin-1-yl, 2-oxopyrrolidin-1-yl, 2-oxo-1H-pyridin-1-yl, 3-oxomorpholin-4-yl, 4-oxo-1 H-pyridin-1-yl, 2,6-dioxopiperidin-1-yl, 2-oxopiperazin-1-yl, 2,5-dioxopyrrolidin-1-yl, 2-oxo-1,3-oxazolidin-3-yl, 3-oxo-2H-pyridazin-2-yl or 2-caprolactam-1-yl, very particularly preferably 2-oxopiperidin-1-yl, 2-oxopyrrolidin-1-yl or 2-caprolactam-1-yl.

The compounds of the formula I may have one or more chiral centres and therefore occur in various stereoisomeric forms. The formula I covers all these forms.

Accordingly, the invention relates in particular to the compounds of the formula I in which at least one of the said radicals has one of the preferred meanings indicated above. Some preferred groups of compounds may be expressed by the following sub-formulae Ia to Ix, which conform to the formula I and in which the radicals not designated in greater detail are as defined under the formula I, but in which

in Ia R¹ is H, A or Ar-alk;

in Ib R² is Ar;

in Ic R¹ is H, alkyl having 1, 2, 3, 4, 5 or 6 carbon atoms, in which 1-5 H atoms may be replaced by F, or benzyl;

in Id R² is phenyl which is monosubstituted or disubstituted by Hal, OH, OA′, COOH, COOA′, CONH₂, CONHNH₂, CH₂NH₂, CH₂NHA′, CH(NH₂)CH₂NH₂, C(═NH)NH₂, C(═NH)NH—COOA′, SO₂NR⁸R⁹,

or

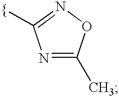

in Ie Z is an unsubstituted alkylene chain (CH₂)ₘ and m is 0 or 1;

in If R³ is H or A;

in Ig R⁴ is H, F or Cl;

in Ih X is phenyl which is monosubstituted or disubstituted by CH₂CH₂NH₂, C(═NH)NH₂, SO₂CH₂R⁶ or SO₂NR⁸R⁹, or an unsubstituted radical of the formula IIa, lIb, IIc or IId 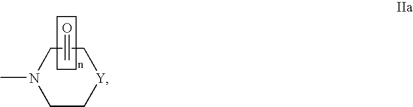 (CH₂)ₚ—SO₂—(CH₂)ₙ—R⁶ IIb,

(CH₂)ₚ—SO₂—NR⁸R⁹ IIc,

(CH₂)ₚ—NH—SO₂—(CH₂)ₙ—R⁶ IId;

in Ii X is phenyl which is monosubstituted or disubstituted by CH₂CH₂NH₂, C(═NH)NH₂, SO₂CH₂R⁶ or SO₂NR⁸R⁹, or an unsubstituted radical of the formula IIa or IId 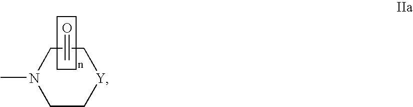 (CH₂)ₚ—NH—SO₂—(CH₂)ₙ—R⁶ IId;

in Ij X is phenyl which is monosubstituted or disubstituted by CH₂CH₂NH₂, C(═NH)NH₂, SO₂A″ or SO₂NH₂, or an unsubstituted radical of the formula IIa or IId 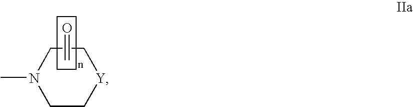 (CH₂)ₚ—NH—SO₂—CH₃ IId,

A″ is alkyl having 1, 2, 3, 4, 5 or 6 carbon atoms,

n is 1 or 2,

p is 1 or 2,

Y is O, NR⁵ or an unsubstituted alkylene chain (CH₂)_{m′},

m′ is 0, 1 or 2;

in Ik R⁵ is H;

in Il T is absent;

in Im R⁶ is H or A;

in In R⁷ is NH₂;

in Io R⁸ is H;

in Ip R⁹ is H, A, benzyl, Het, Q¹ or Q²;

in Iq R⁸ and R⁹, together with the nitrogen to which they are bonded, are pyrrolidinyl, piperidinyl, piperazinyl, morpholinyl, tetrahydropyrimidinyl, dihydropyridinyl or dihydroimidazolyl;

in Ir Ar is phenyl which is monosubstituted by Hal, OH, OA′, COOH, COOA′, CONH₂, CONHNH₂, CH₂NH₂, CH₂CH₂NH₂, CH₂NHA′, CH(NH₂)CH₂NH₂, C(═NH)NH₂, C(═NH)NH—COOA′, SO₂A′, SO₂NR⁸R⁹,

or

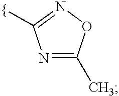

in Is Ar′ is phenyl;

in It U is an unsubstituted radical of the formula IIa or IId 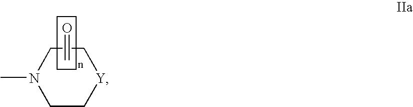 (CH₂)ₚ—NH—SO₂—(CH₂)ₙ—R⁶ IId;

in Iu U is an unsubstituted radical of the formula IIa or IId 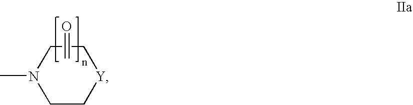 (CH₂)ₚ—NH—SO₂—CH₃ IId,

n is 1 or 2,

p is 1 or 2,

Y is O, NR⁵ or an unsubstituted alkylene chain (CH₂)ₘ,

R⁵ is H,

m is 0, 1 or 2;

in Iv Q² is pyrrolidinyl, piperidinyl, piperazinyl, morpholinyl, tetrahydropyrimidinyl, dihydropyridinyl or dihydroimidazolyl;

in Iw the radical of the formula IIa is:

morpholin-4-yl, 2-oxopiperidin-1-yl, 2-oxopyrrolidin-1-yl, 2-oxo-1H-pyridin-1-yl, 3-oxomorpholin-4-yl, 4-oxo-1H-pyridin-1-yl, 2,6-dioxopiperidin-1-yl, 2-oxopiperazin-1-yl, 2,5-dioxopyrrolidin-1-yl, 2-oxo-1,3-oxazolidin-3-yl, 3-oxo-2H-pyridazin-2-yl or 2-caprolactam-1-yl;

in Ix R¹ is H, alkyl having 1, 2, 3, 4, 5 or 6 carbon atoms, in which 1-5 H atoms may be replaced by F, or benzyl,

R² is phenyl which is monosubstituted or disubstituted by Hal, OH, OA′, COOH, COOA′, CONH₂, CONHNH₂, CH₂NH₂, CH₂NHA′, CH(NH₂)CH₂NH₂, C(═NH)NH₂, C(═NH)NH—COOA′, SO₂NR⁸R⁹,

or

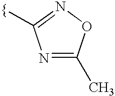

Z is an unsubstituted alkylene chain (CH₂)ₘ,

m is 0 or 1,

R³ is H or A,

R⁴ is H, F or Cl,

X is phenyl which is monosubstituted or disubstituted by CH₂CH₂NH₂, C(═NH)NH₂, SO₂A″ or SO₂NH₂, or an unsubstituted radical of the formula IIa or IId 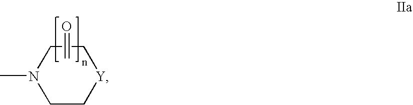 (CH₂)ₚ—NH—SO₂—CH₃ IId,

A″ is alkyl having 1, 2, 3, 4, 5 or 6 carbon atoms,

n is 1 or 2,

p is 1 or 2,

Y is O, NR⁵ or an unsubstituted alkylene chain (CH₂)_{m′},

m′ is 0, 1 or 2;

and pharmaceutically tolerated salts, solvates and stereoisomers thereof.

The compounds of the formula I and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

If desired, the starting materials can also be formed in situ so that they are not isolated from the reaction mixture, but instead are immediately converted further into the compounds of the formula I.

Compounds of the formula I can preferably be obtained by liberating compounds of the formula I from one of their functional derivatives by treatment with a solvolysing or hydrogenolysing agent.

Preferred starting materials for the solvolysis or hydrogenolysis are those which conform to the formula 1, but contain corresponding protected amino and/or hydroxyl groups instead of one or more free amino and/or hydroxyl groups, preferably those which carry an amino-protecting group instead of an H atom bonded to an N atom, in particular those which carry an R′—N group, in which R′ is an amino-protecting group, instead of an HN group, and/or those which carry a hydroxyl-protecting group instead of the H atom of a hydroxyl group, for example those which conform to the formula I, but carry a —COOR″ group, in which R″ is a hydroxyl-protecting group, instead of a —COOH group. Preferred starting materials are also the oxadiazole derivatives, which can be converted into the corresponding amidino compounds.

The amidino group can be liberated from its oxadiazole derivative by, for example, treatment with hydrogen in the presence of a catalyst (for example Raney nickel). Suitable solvents are those indicated below, in particular alcohols, such as methanol or ethanol, organic acids, such as acetic acid or propionic acid, or mixtures thereof. The hydrogenolysis is generally carried out at temperatures between about 0 and 100° and pressures between about 1 and 200 bar, preferably at 20-30° (room temperature) and 1-10 bar.

The oxadiazole group is introduced, for example, by reaction of the cyano compounds with hydroxylamine and reaction with phosgene, dialkyl carbonate, chloroformic acid esters, N,N′-carbonyldiimidazole or acetic anhydride.

It is also possible for a plurality of—identical or different—protected amino and/or hydroxyl groups to be present in the molecule of the starting material. If the protecting groups present are different from one another, they can in many cases be cleaved off selectively.

The term “amino-protecting group” is known in general terms and relates to groups which are suitable for protecting (blocking) an amino group against chemical reactions, but which are easy to remove after the desired chemical reaction has been carried out elsewhere in the molecule. Typical of such groups are, in particular, unsubstituted or substituted acyl, aryl, aralkoxymethyl or aralkyl groups. Since the amino-protecting groups are removed after the desired reaction (or reaction sequence), their type and size are furthermore not crucial; however, preference is given to those having 1-20, in particular 1-8, carbon atoms. The term “acyl group” is to be understood in the broadest sense in connection with the present process. It includes acyl groups derived from aliphatic, araliphatic, aromatic or heterocyclic carboxylic acids or sulfonic acids, and, in particular, alkoxycarbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and tolyl; aryloxyalkanoyl, such as POA; alkoxycarbonyl, such as methoxycarbonyl, ethoxycarbonyl, 2,2,2-trichloroethoxycarbonyl, BOC (tert-butoxycarbonyl) and 2-iodoethoxycarbonyl; aralkbxycarbonyl, such as CBZ (“carbobenzoxy”), 4-methoxybenzyloxycarbonyl and FMOC; and arylsulfonyl, such as Mtr. Preferred amino-protecting groups are BOC and Mtr, furthermore CBZ, Fmoc, benzyl and acetyl.

The term “hydroxyl-protecting group” is likewise known in general terms and relates to groups which are suitable for protecting a hydroxyl group against chemical reactions, but are easily removable after the desired chemical reaction has been carried out elsewhere in the molecule. Typical of such groups are the above-mentioned unsubstituted or substituted aryl, aralkyl or acyl groups, furthermore also alkyl groups. The nature and size of the hydroxyl-protecting groups are not crucial since they are removed again after the desired chemical reaction or reaction sequence; preference is given to groups having 1-20, in particular 1-10, carbon atoms. Examples of hydroxyl-protecting groups are, inter alia, benzyl, 4-methoxybenzyl, p-nitrobenzoyl, p-toluenesulfonyl, tert-butyl and acetyl, where benzyl and tert-butyl are particularly preferred.

The compounds of the formula I are liberated from their functional derivatives—depending on the protecting group used—for example using strong acids, advantageously using TFA or perchloric acid, but also using other strong inorganic acids, such as hydrochloric acid or sulfuric acid, strong organic carboxylic acids, such as trichloroacetic acid, or sulfonic acids, such as benzene- or p-toluenesulfonic acid. The presence of an additional inert solvent is possible, but is not always necessary. Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols, such as methanol, ethanol or isopropanol, and water. Mixtures of the above-mentioned solvents are furthermore suitable. TFA is preferably used in excess without addition of a further solvent, and perchloric acid is preferably used in the form of a mixture of acetic acid and 70% perchloric acid in the ratio 9:1. The reaction temperatures for the cleavage are advantageously between about 0 and about 50°, preferably between 15 and 30° (room temperature).

The BOC, Obut and Mtr groups can, for example, preferably be cleaved off using TFA in dichloromethane or using approximately 3 to 5N HCl in dioxane at 15-30°, and the FMOC group can be cleaved off using an approximately 5 to 50% solution of dimethylamine, diethylamine or piperidine in DMF at 15-30°.

Protecting groups which can be removed hydrogenolytically (for example CBZ, benzyl or the liberation of the amidino group from its oxadiazole derivative) can be cleaved off, for example, by treatment with hydrogen in the presence of a catalyst (for example a noble-metal catalyst, such as palladium, advantageously on a support, such as carbon). Suitable solvents here are those indicated above, in particular, for example, alcohols, such as methanol or ethanol, or amides, such as DMF. The hydrogenolysis is generally carried out at temperatures between about 0 and 100° and pressures between about 1 and 200 bar, preferably at 20-30° and 1-10 bar. hydrogenolysis of the CBZ group succeeds well, for example, on 5 to 10% Pd/C in methanol or using ammonium formate (instead of hydrogen) on Pd/C in methanol/DMF at 20-30°.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, trifluoromethylbenzene, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone (NMP) or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

A cyano group is converted into an amidino group by reaction with, for example, hydroxylamine followed by reduction of the N-hydroxyamidine using hydrogen in the presence of a catalyst, such as, for example, Pd/C. In order to prepare an amidine of the formula I, it is also possible to adduct ammonia onto a nitrile. The adduction is preferably carried out in a number of steps by, in a manner known per se, a) converting the nitrile into a thioamide using H₂S, converting the thioamide into the corresponding S-alkyl-imidothioester using an alkylating agent, for example CH₃l, and reacting the thioester in turn with NH₃ to give the amidine, b) converting the nitrile into the corresponding imidoester using an alcohol, for example ethanol in the presence of HCl, and treating the imidoester with ammonia (Pinner synthesis), or c) reacting the nitrite with lithium bis(trimethylsilyl)amide, and subsequently hydrolysing the product.

Esters can be saponified, for example, using acetic acid or using NaOH or KOH in water, water/THF or water/dioxane, at temperatures between 0 and 100°.

Free amino groups can furthermore be acylated in a conventional manner using an acid chloride or anhydride or alkylated using an unsubstituted or substituted alkyl haIIde, or reacted with CH₃—C(═NH)—Oet, advantageously in an inert solvent, such as dichloromethane or THF and/or in the presence of a base, such as triethylamine or pyridine, at temperatures between −60 and +30°.

If desired, the starting materials can also be formed in situ so that they are not isolated from the reaction mixture, but instead are immediately converted further into the compounds of the formula I.

Compounds of the formula I in which free NH and/or OH groups are in protected form can preferably be obtained by reacting compounds of the formula II with compounds of the formula III or by reacting compounds of the formula IV with compounds of the formula V. The reaction is generally carried out in an inert solvent, in the presence of an acid-binding agent, preferably an alkali or alkaline earth metal hydroxide, carbonate or bicarbonate, or in the presence of another salt of a weak acid of the alkali or alkaline earth metals, preferably of potassium, sodium, calcium or caesium. The addition of an organic base, such as triethylamine, dimethylaniline, pyridine or quinoline, may also be favourable. Depending on the conditions used, the reaction time is between a few minutes and 14 days, and the reaction temperature is between about 0° and 150°, normally between 20° and 130°.

Examples of suitable inert solvents are water; hydrocarbons, such as hexane, petroleum ether, benzene, toluene or xylene; chlorinated hydrocarbons, such as trichloroethylene, 1,2-dichloroethane, carbon tetrachloride, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

The starting compounds of the formulae II, III, IV and V are generally known. If they are novel, however, they can be prepared by methods known per se.

In the compounds of the formula II and V, L is preferably Cl, Br, I or a reactively modified OH group, such as, for example, an activated ester, an imidazolide or alkylsulfonyloxy having 1-6 carbon atoms (preferably methylsulfonyloxy or trifluoromethylsulfonyloxy) or arylsulfonyloxy having 6-10 carbon atoms (preferably phenyl- or p-tolylsulfonyloxy).

Compounds of the general formula II are prepared by processes known per se by reacting a monoalkyl oxalate of the general formula VI

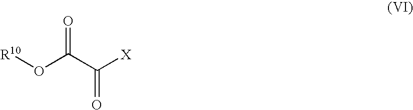

in which X is as defined above, and R¹⁰ is a lower alkyl radical, in particular the methyl or ethyl radical, with an amine of the general formula V, and subsequently hydrolysing the ester group by processes known per se.

Compounds of the general formula IV are prepared by processes known per se by reacting a monoalkyl oxalate of the general formula VI with an amine of the general formula III, and subsequently hydrolysing the ester group by processes known per se.

Compounds of the general formula II or IV in which L is chlorine are obtained from compounds of the general formula II or IV in which L is the OH group by processes known per se by reaction with thionyl chloride or oxalyl chloride.

A base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable salts. Thus, it is possible to use inorganic acids, for example sulfuric acid, nitric acid, hydrohalic acids, such as hydrochloric acid or hydrobromic acid, phosphoric acids, such as orthophosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids, and laurylsulfuric acid. Salts with physiologically unacceptable acids, for example picrates, can be used for the isolation and/or purification of the compounds of the formula I.

On the other hand, compounds of the formula I can be converted into the corresponding metal salts, in particular alkali metal or alkaline earth metal salts, or into the corresponding ammonium salts using bases (for example sodium hydroxide, potassium hydroxide, sodium carbonate or potassium carbonate). It is also possible to use physiologically acceptable organic bases, such as, for example, ethanolamine.

Compounds of the formula I according to the invention may be chiral owing to their molecular structure and may accordingly occur in various enantiomeric forms. They can therefore exist in racemic or in optically active form.

Since the pharmaceutical activity of the racemates or stereoisomers of the compounds according to the invention may differ, it may be desirable to use the enantiomers. In these cases, the end product or even the intermediates can be separated into enantiomeric compounds by chemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitable N-protected amino acids (for example N-benzoylproline or N-benzenesulfonylproline), or the various optically active camphorsulfonic acids. Also advantageous is chromatographic enantiomer resolution with the aid of an optically active resolving agent (for example dinitrobenzoylphenylglycine, cellulose triacetate or other derivatives of carbohydrates or chirally derivatised methacrylate polymers immobilised on silica gel). Suitable eluents for this purpose are aqueous or alcoholic solvent mixtures, such as, for example, hexane/isopropanol/acetonitrile, for example in the ratio 82:15:3.

The invention furthermore relates to the use of the compounds of the formula I and/or their physiologically acceptable salts for the preparation of pharmaceutical preparations, in particular by non-chemical methods. They can be converted here into a suitable dosage form together with at least one solid, liquid and/or semi-liquid excipient or assistant and, if desired, in combination with one or more further active ingredients.

The invention furthermore relates to pharmaceutical preparations comprising at least one compound of the formula I and/or one of its physiologically acceptable salts.

These preparations can be used as medicaments in human or veterinary medicine. Suitable excipients are organic or inorganic substances which are suitable for enteral (for example oral), parenteral or topical administration and do not react with the novel compounds, for example water, vegetable oils, benzyl alcohols, alkylene glycols, polyethylene glycols, glycerol triacetate, gelatin, carbohydrates, such as lactose or starch, magnesium stearate, talc or vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders or also as nasal sprays. The novel compounds may also be lyophilised and the resultant lyophilisates used, for example, to prepare injection preparations. The preparations indicated may be sterilised and/or comprise assistants, such as lubricants, preservatives, stabilisers and/or wetting agents, emulsifying agents, salts for modifying the osmotic pressure, buffer substances, colorants and flavours and/or a plurality of further active ingredients, for example one or more vitamins. The injection medium used is preferably water containing the conventional additives in injection solutions, such as stabilisers, solubilisers and buffers. Additives of this type are, for example, tartrate and citrate buffer, ethanol, complexing agents (such as ethylenediaminetetraacetic acid and non-toxic salts thereof), high-molecular-weight polymers (such as liquid polyethylene oxide) for regulating the viscosity. Liquid excipients for injection solutions must be sterile and are. preferably packaged in ampoules. Solid excipients are, for example, starch, lactose, mannitol, methylcellulose, talc, highly disperse silicic acids, relatively high-molecular-weight fatty acids (such as stearic acid), gelatine, agar-agar, calcium phosphate, magnesium stearate, animal and vegetable fats, solid high-molecular-weight polymers (such as polyethylene glycols).

The compounds of the formula I and their physiologically acceptable salts can be used for combating and preventing thromboembolic disorders, such as thrombosis, myocardial infarction, arteriosclerosis, inflammation, apoplexia, angina pectoris, restenosis after angioplasty and claudicatio intermittens.

In general, the substances according to the invention are preferably administered in doses between about 1 and 500 mg, in particular between 5 and 100 mg, per dosage unit. The daily dose is preferably between about 0.02 and 10 mg/kg of body weight. However, the specific dose for each patient depends on a wide variety of factors, for example on the efficacy of the specific compound employed, on the age, body weight, general state of health, sex, on the diet, on the time and method of administration, on the excretion rate, medicament combination and severity of the particular illness to which the therapy applies. Oral administration is preferred.

Above and below, all temperatures are given in ° C. In the following examples, “conventional work-up” means that water is added if necessary, the pH is adjusted, if necessary, to between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation. Rf values on silica gel; eluent:ethyl acetate/methanol 9:1.

Mass spectrometry (MS): EI (electron impact ionisation) M⁺

FAB (fast atom bombardment) (M+H)⁺

ESI (electrospray ionisation) (M+H)⁺ (unless specified otherwise)

### EXAMPLE 1

### N-(3-amidinobenzyl)-N-isobutyl-N′-(2′-sulfamoylbiphenyl-4-yl)oxalamide

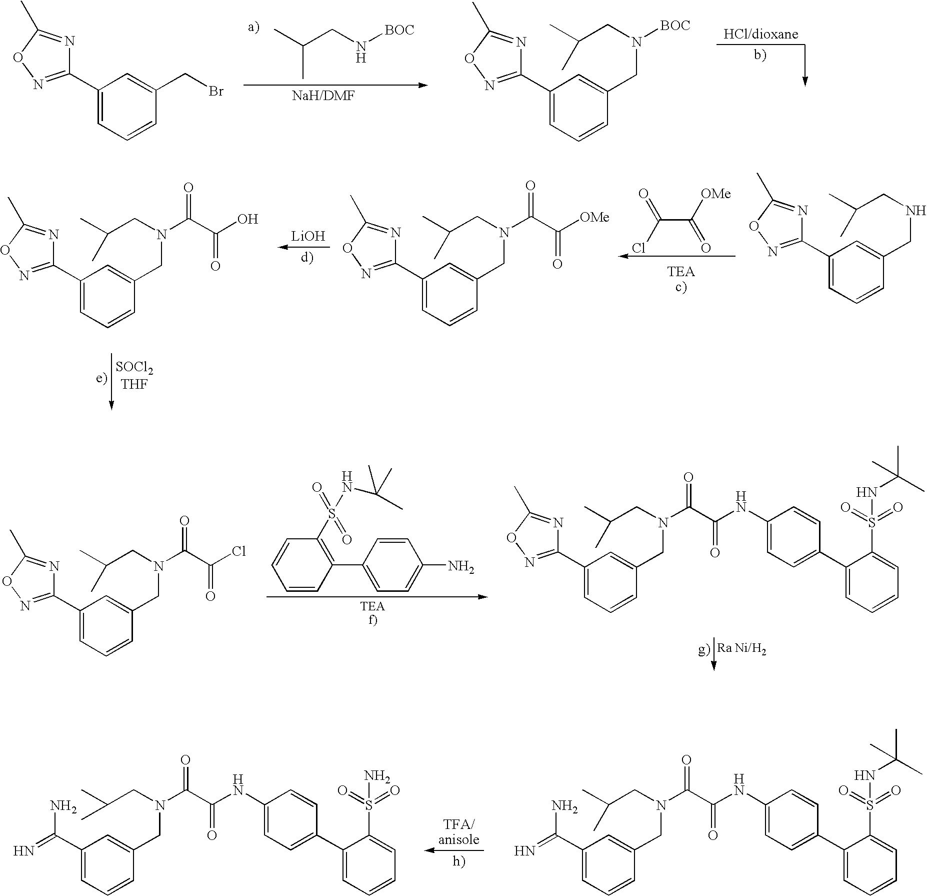

1a) A solution of 5 g (28.8 mmol) of tert-butyl isobutylcarbamate in 10 ml of DMF is added dropwise under a nitrogen atmosphere to a suspension of 1.27 g (31.74 mmol) of sodium hydride in 40 ml of dry dimethylformamide (DMF), and the reaction mixture is then stirred at room temperature for 8 hours. A solution of 7.3 g (28.8 mmol) of 3-(3-bromomethylphenyl)-5-methyl-1,2,4-oxadiazole in 20 ml of DMF is subsequently added, and the mixture is stirred at room temperature for a further 10 hours. The DMF is then distilled off under reduced pressure, the residue is taken up in 30 ml of water, and the aqueous solution is extracted twice with 20 ml of ethyl acetate each time. After the combined extracts have been dried over sodium sulfate and the solvent has been stripped off, the residue is purified by column chromatography on silica gel (methanol/methylene chloride: 0.1/9.9), giving 2.3 g of tert-butyl isobutyl [3-(5-methyl-1,2,4-oxadiazol-3-yl)benzyl]carbamate; ESI 346.

1b) 20 ml of 4N HCl in dioxane are added with stirring to the solution of 2.2 g (6.0 mmol) of tert-butyl isobutyl [3-(5-methyl-1,2,4-oxadiazol-3-yl)benzyl]carbamate in 30 ml of dioxane. After a reaction time of 7 hours, the dioxane is removed under reduced pressure, giving 2.0 g of isobutyl[3-(5-methyl-1,2,4-oxadiazol-3-yl)benzyl]amine as the HCl salt; ESI 246.

1c) The solution of 0.6 ml (7.0 mmol) of monomethyl oxalate chloride in 5 ml of THF is added dropwise at room temperature to the solution of 2.0 g (8.0 mmol) of isobutyl[3-(5-methyl-1,2,4-oxadiazol-3-yl)benzyl]amine HCl salt and 1 ml of triethylamine in 40 ml of absolute tetrahydrofuran (THF). The reaction mixture is subsequently stirred at room temperature for a further two hours and then evaporated to dryness, and the residue is taken up in 20 ml of ethyl acetate. This solution is then washed three times with 20 ml of water each time, and the organic phase is dried over sodium sulfate. The stripping-off of the solvent gives 2.3 g of methyl N-isobutyl-N-[3-(5-methyl-1,2,4-oxadiazol-3-yl)benzyl]oxalamidate as a pale-yellow oil; ESI 332.

1d) 0.42 g (18 mmol) of lithium hydroxide is added to the solution of 4.5 g (14 mmol) of N-isobutyl-N-[3-(5-methyl-1,2,4-oxadiazol-3-yl)benzyl]oxalamidate in 120 ml of tetrahydrofuran (THF) and 40 ml of water, and the mixture is stirred at room temperature for 16 hours. The THF is subsequently stripped off under reduced pressure, and the aqueous solution is acidified with 2N hydrochloric acid and extracted three times with 20 ml of ethyl acetate each time. Drying of the combined organic phases over sodium sulfate and stripping-off of the solvent gives 3.75 g of N-isobutyl-N-[3-(5-methyl-1,2,4-oxadiazol-3-yl)benzyl]oxalamic acid as a yellow oil; FAB [M+Li]⁺ 330.

1e), 1f) One drop of dimethylformamide and then 2.26 ml of thionyl chloride are added to the solution of 10.0 g (31.5 mmol) of N-isobutyl-N-[3-(5-methyl-1,2,4-oxadiazol-3-yl)benzyl]oxalamidate in 200 ml of absolute tetrahydrofuran, and the mixture is stirred at room temperature for 4 hours. 4.1 ml of triethylamine and 9.98 g (31.47 mmol) of N-tert-butyl-4′-aminobiphenyl-2-sulfonamide are subsequently added, and the reaction mixture is stirred at room temperature for 14 hours. The solvent is then stripped off under reduced pressure, the residue is taken up in 400 ml of methylene chloride, and the solution is washed three times with 100 ml of water each time. After the organic phase has been dried over sodium sulfate and the solvent has been stripped off, the residue is chromatographed on silica gel (petroleum ether/ethyl acetate: 1/1), giving 5.2 g of N-(2′-tert-butylsulfamoylbiphenyl-4-yl)-N′-isobutyl-N′-[3-(5-methyl-1,2,4-oxadiazol-3-yl)benzyl]oxalamide; ESI 604.

1g) 7 g of Raney nickel are added to the solution of 5.1 g (8.45 mmol) of N-(2′-tert-butylsulfamoylbiphenyl-4-yl)-N′-isobutyl-N′-[3-(5-methyl-1,2,4-oxadiazol-3-yl)benzyl]oxalamide in 200 ml of methanol, 2.5 ml of acetic acid and 2.5 ml of water, and the mixture is hydrogenated at room temperature and atmospheric pressure for 15 hours. The reaction mixture is then filtered, and the filtrate is evaporated to dryness. The oily residue obtained in this way is crystallised by addition of diethyl ether, giving 5 g of N-(2′-tert-butylsulfamoylbiphenyl-4-yl)-N′-(3-amidinobenzyl)-N′-isobutyloxalamide acetate as a grey amorphous solid; FAB 564.

Affinity to receptors:

IC₅₀ (Xa)=0.3 μM.

1h) 0.5 ml of anisole is added to the solution of 5 g (8.87 mmol) of N-(2′-tert-butylsulfamoylbiphenyl-4-yl)-N′-(3-amidinobenzyl)-N′-isobutyloxalamide in 80 ml of trifluoroacetic acid, and the mixture is stirred at room temperature for 16 hours. The reaction solution is subsequently evaporated to dryness, and the residue is crystallised by addition of diethyl ether, giving 4.3 g of N-(3-amidinobenzyl)-N-isobutyl-N′-(2′-sulfamoylbiphenyl-4-yl)oxalamide trifluoroacetate; FAB 508.

Affinity to receptors:

IC₅₀ (Xa)=10 nM.

### EXAMPLE 2

### N-(3-amidinobenzyl)-N′-(2′-sulfamoylbiphenyl-4-yl)oxalamide

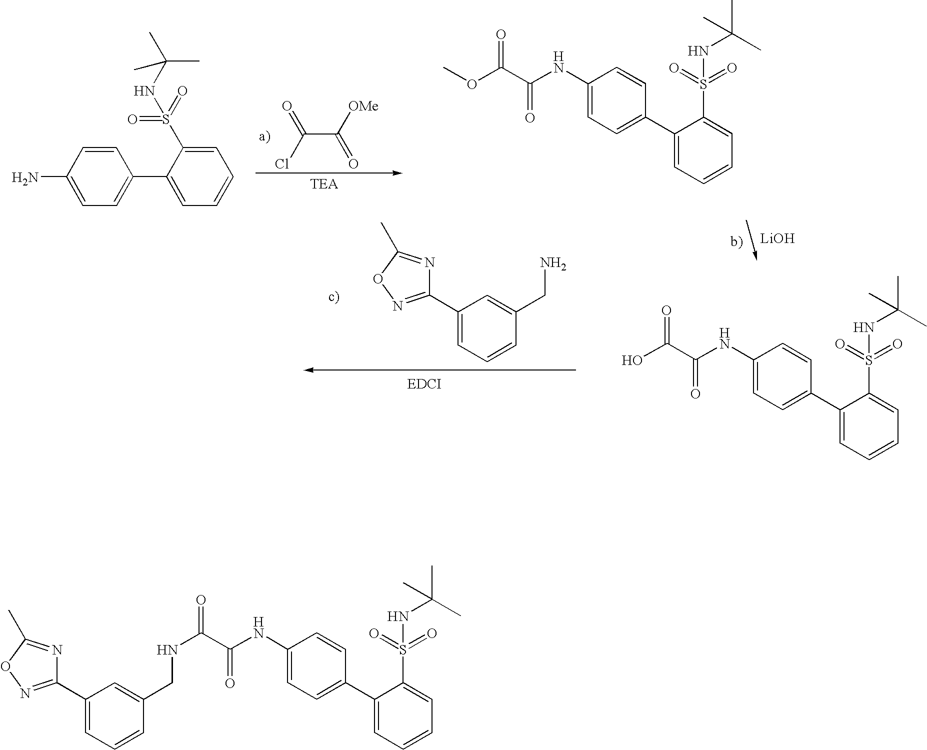

2a) A solution of 1.2 g (10 mmol) of monomethyl oxalate chloride in 5 ml of THF is added dropwise at room temperature to the solution of 3.0 g (10 mmol) of N-tert-butyl-4′-aminobiphenyl-2-sulfonamide and 1.35 ml of triethylamine (TEA) in 75 ml of absolute tetrahydrofuran (THF). The reaction mixture is subsequently stirred at room temperature for four hours and then evaporated to dryness, and the residue is taken up in 20 ml of ethyl acetate. This solution is then washed three times with 20 ml of water each time, and the organic phase is dried over sodium sulfate. After the solvent has been stripped off, the residue is recrystallised from diethyl ether, giving 3.9 g of methyl N-(2′-tert-butylsulfamoylbiphenyl-4-yl)oxalamidate as a white powder; m.p. 132.2°.

2b) A solution of 120 mg of lithium hydroxide (LiOH) in 5 ml of water is added to the solution of 1.95 g (5 mmol) of methyl N-(2′-tert-butylsulfamoylbiphenyl-4-yl)oxalamidate in 40 ml of methanol, and the reaction mixture is stirred at room temperature for 16 hours. The solvent is subsequently stripped off under reduced pressure, and the residue is taken up in 20 ml of water. After the aqueous solution has been acidified with 2N hydrochloric acid, the precipitate is filtered off and washed with water, giving 1.8 g of N-(2′-tert-butylsulfamoylbiphenyl-4-yl)oxalamic acid as a pale-grey powder; m.p. 159.3°.

2c) 0.26 g of N-(3-dimethylaminopropyl)-N′-ethylcarbodiimide hydrochloride is added to the solution of 0.3 g (1.33 mmol) of 3-(5-methyl-1,2,4-oxadiazol-3-yl)benzylamine hydrochloride, 0.15 ml of N-methylmorpholine, 0.18 g of 1-hydroxybenzotriazole and 0.5 g (1.33 mmol) of N-(2′-tert-butylsulfamoylbiphenyl-4-yl)oxalamic acid in 15 ml of DMF, and the reaction mixture is stirred at room temperature for 15 hours. The mixture is subsequently evaporated under reduced pressure, 20 ml of 5% sodium hydrogencarbonate solution are added to the residue, and the precipitate is filtered off with suction and washed with water. Recrystallisation from ethanol gives 0.41 g of N-(2′-tert-butylsulfamoylbiphenyl-4-yl)-N′-[3-(5-methyl-1,2,4-oxadiazol-3-yl)benzyl]oxalamide as slightly yellowish crystals; FAB 548.

2d) Analogously to 1g)-1h), reaction of 2c) gives N-(3-amidinobenzyl)-N′-(2′-sulfamoylbiphenyl-4-yl)oxalamide as the trifluoroacetate salt; FAB 452.

### EXAMPLE 3

### N-(3-amidinobenzyl)-N-isobutyl-N′-(2′-methanesulfonylbiphenyl-4-yl)oxalamide

Reaction of the acid 1d) and 2′-methanesulfonylbiphenyl-4-ylamine analogously to 1e)-1h) gives the title compound as the acetate. MALDI-MS: 507.

Affinity to receptors:

IC₅₀ (Xa)=9.6 nM.

### EXAMPLE 4

### Methyl [imino-(3-{[isobutyl-(2′-methanesulfonylbiphenyl-4-ylaminooxalyl)amino]methyl}phenyl)methyl]carbamate

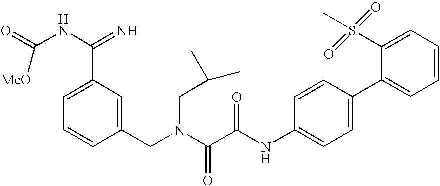

The mixture of 115 mg (0.2 mmol) of N-(3-amidinobenzyl)-N-isobutyl-N′-(2′-methanesulfonylbiphenyl-4-yl)oxalamide (Ex.1) in 3 ml of methylene chloride, 3 ml of water and 0.2 ml of 1N sodium hydroxide solution is stirred at room temperature for 45 minutes with 20 mg of methyl chloroformate and at pH 9. The organic phase is subsequently separated off, and the aqueous phase is washed by shaking twice with 5 ml of methylene chloride each time. After the combined organic phases have been dried over sodium sulfate and the solvent has been stripped off, the crude product is purified by column chromatography on silica gel (methylene chloride/methanol: 99/1), giving 44 mg of the title compound; FAB 565.

Affinity to receptors:

IC₅₀ (Xa)=3.5 μM.

### EXAMPLE 5

### N-(3-amidinobenzyl)-N′-(4′-amidinobiphenyl-4-yl)-N-isobutyloxalamide

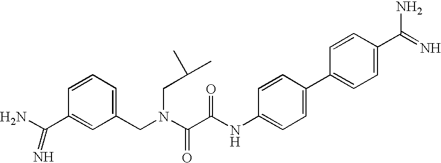

5a) Reaction of the acid 1d) and 4′-aminobiphenyl-4-carbonitrile analogously to 1e)-1f) gives the compound N-(4′-cyanobiphenyl-4-yl)-N′-isobutyl-N′-[3-(5-methyl-1,2,4-oxadiazol-3-yl)benzyl]oxalamide as pale-grey crystals; FAB 494.

5b) Hydrogen chloride is passed into the solution of 300 mg of N-(4′-cyanobiphenyl-4-yl)-N′-isobutyl-N′-[3-(5-methyl-1,2,4-oxadiazol-3-yl)-benzyl]oxalamide in 40 ml of ethanol at 5° C. with stirring until the solution is saturated (2 hours). The reaction solution is subsequently stirred overnight at room temperature and then evaporated to dryness under reduced pressure, and the residue is taken up in 40 ml of ethanol. 375 mg of ammonium acetate are then added to this solution, and the reaction solution is refluxed for 16 hours. After cooling, the solvent is stripped off under reduced pressure, 10 ml of water are added to the residue, and the aqueous solution is rendered alkaline with saturated sodium hydrogencarbonate solution and extracted three times with 10 ml of methylene chloride each time. Drying of the combined extracts over sodium sulfate and stripping-off of the solvent gives 140 mg of N-(4′-amidinobiphenyl-4-yl)-N′-isobutyl-N′-[3-(5-methyl-1,2,4-oxadiazol-3-yl)benzyl]oxalamide as an amorphous powder; FAB 511.

5c) Reaction of 110 mg (0.215 mmol) of N-(4′-amidinobiphenyl-4-yl)-N′-isobutyl-N′-[3-(5-methyl-1,2,4-oxadiazol-3-yl)benzyl]oxalamide analogously to 1g) gives 60 mg of the title compound as the diacetate salt; FAB 471.

Affinity to receptors:

IC₅₀ (Xa)=1.0 μM.

### EXAMPLE 6

### N-(4′-Aminomethylbiphenyl-4-yl)-N′-(3-amidinobenzyl)-N′-isobutyloxalamide

Reaction of 180 mg (0.36 mmol) of N-(4′-cyanobiphenyl-4-yl)-N′-isobutyl-N′-[3-(5-methyl-1,2,4-oxadiazol-3-yl)benzyl]oxalamide 5a) and 2 g of Raney nickel analogously to 1g) gives 74 mg of the title compound as the acetate salt; FAB 458.

Affinity to receptors:

IC₅₀ (Xa)=1.5 μM.

### EXAMPLE 7

### 3-{[Isobutyl-(2′-sulfamoylbiphenyl-4-ylaminooxalyl)amino]methyl }benzoic acid

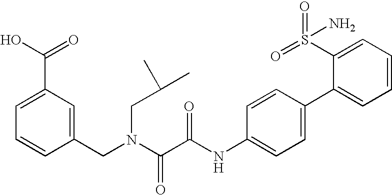

7a) Reaction of methyl 3-bromomethylbenzoate (Kotani, T.; Chem.Pharm.Bull.; EN; 45; 2; 1997; 297-304) and tert-butyl isobutylcarbamate by means of sodium hydride analogously to Example 1a)-1b) gives methyl 3-(isobutylaminomethyl)benzoate as the trifluoroacetate salt; ESI 222.

7b) Reaction of the acid 2b), thionyl chloride and methyl 3-(isobutylaminomethyl)benzoate 7a) analogously to Example 1e)-1f) gives the compound methyl 3-{[(2′-tert-butylsulfamoylbiphenyl-4-ylaminooxalyl)isobutylamino]methyl}benzoate as an amorphous powder; FAB 580.

7c) Reaction of methyl 3-{[(2′-tert-butylsulfamoylbiphenyl-4-ylaminooxalyl)isobutylamino]methyl}benzoate 7b) and trifluoroacetic acid analogously to Example 1h) gives methyl 3-{[isobutyl-(2′-sulfamoylbiphenyl-4-ylaminooxaiyl)amino]methyl}benzoate as a crystalline substance; FAB 524.

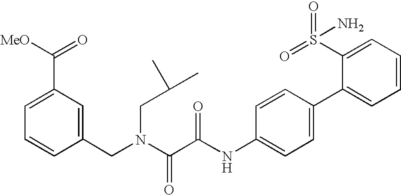

7d) Reaction of the methyl ester 7c) analogously to Example 1d) gives the title compound as a pale-yellow powder; FAB 510.

Affinity to receptors:

IC50 (Xa)=6.0 μM.

### EXAMPLE 8

### N-(3-amidinophenyl)-N-isobutyl-N′-(2′-sulfamoylbiphenyl-4-yl)oxalamide

8a) Reaction of the acid 2b), thionyl chloride and isobutyl-[3-(5-methyl-1,2,4-oxadiazol-3-yl)phenyl]amine analogously to Example 1e)-1f) gives the compound N-(2′-tert-butylsulfamoylbiphenyl-4-yl)-N′-isobutyl-N′-[3-(5-methyl-1,2,4-oxadiazol-3-yl)phenyl]oxalamide as a viscous colourless oil; ESI 590.

9b) Reaction of compound 8a) analogously to Example 1g)-1h) gives the title compound as pale-pink crystals; ESI 494.

### EXAMPLE 9

### N-(3-amidinophenyl)-N-isobutyl-N′-(2′-sulfamoylbiphenyl-4-yl)oxalamide

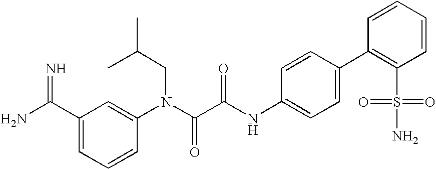

9a) Reaction of the acid 2b), thionyl chloride and isobutyl-[3-(5-methyl-1,2,4-oxadiazol-3-yl)phenyl]amine analogously to Example 1e)-1f) gives the compound N-(2′-tert-butylsulfamoylbiphenyl-4-yl)-N′-isobutyl-N′-[3-(5-methyl-1,2,4-oxadiazol-3-yl)phenyl]oxalamide as a viscous colourless oil, ESI 590.

9b) Reaction of compound 9a) analogously to Example 1g)-1h) gives the title compound as pale-pink crystals, ESI 494.

### EXAMPLE 10

### N-(3-Hydrazinocarbonylbenzyl)-N-isobutyl-N′-(2′-sulfamoylbiphenyl-4-yl)oxalamide

10a) Reaction of the methyl ester 7b) and lithium hydroxide analogously to 1d) gives the compound 3-{[(2′-tert-butylsulfamoylbiphenyl-4-ylaminooxalyl)isobutylamino]methyl}benzoic acid as colourless crystals, MALDI-MS 588.

10b) The mixture of 282 mg of acid 10a) and 20 ml of thionyl chloride is refluxed for four hours and subsequently evaporated to dryness under reduced pressure. The residue is then taken up in 5 ml of tetrahydrofuran, and 130 mg of hydrazine hydrate in 20 ml of tetrahydrofuran are added to the solution. The reaction mixture is stirred at room temperature for one hour and then evaporated to dryness, the residue is taken up in 5 ml of methylene chloride, and the methylene chloride solution is washed three times with 5 ml of water each time. After the organic phase has been dried over sodium sulfate and the solvent has been stripped off, the crude product is purified by column chromatography on silica gel (methylene chloride/methanol: 97/3), giving 90 mg of N-(2′-tert-butylsulfamoylbiphenyl-4-yl)-N′-(3-hydrazinocarbonylbenzyl)-N′-isobutyloxalamide, ESI 580.

10c) Reaction of 10b) and trifluoroacetic acid analogously to Example 1h) gives the title compound as colourless crystals, ESI 524.

### EXAMPLE 11

### N-Benzyl-N-(3-amidinobenzyl)-N′-(2′-sulfamoylbiphenyl-4-yl)oxalamide

11a) Reaction of 3-(3-bromomethylphenyl)-5-methyl-1,2,4-oxadiazole and tert-butyl benzylcarbamate analogously to Example 1a)-1b) gives the compound benzyl[3-(5-methyl-1,2,4-oxadiazol-3-yl)benzyl]amine as a yellow oil, ESI 280.

11b) Reaction of the acid 2b) and the amine 11a) analogously to 2c) gives N-benzyl-N′-(2′-tert-butylsulfamoylbiphenyl-4-yl)-N-[3-(5-methyl-1,2,4-oxadiazol-3-yl)benzyl]oxalamide as a grey solid, ESI 638.

11c) Reaction of 11b) analogously to Example 1g)-1h) gives the title compound as white crystals, ESI 542.

### EXAMPLE 12

### Ethyl [imino-(3-{[isobutyl-(2′-sulfamoylbiphenyl-4-ylaminooxalyl)amino]methyl}phenyl)methyl]carbamate

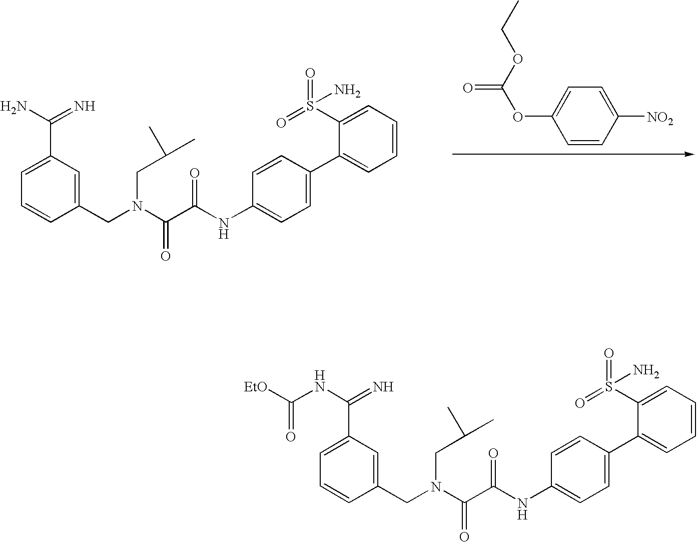

The solution of 311 mg (0.5 mmol) of N-(3-amidinobenzyl)-N-isobutyl-N′-(2′-methanesulfonylbiphenyl-4-yl)oxalamide (Ex.1) and 0.2 ml of triethylamine in 20 ml of dimethylformamide is stirred at 60° C. for 14 hours with 110 mg (0.52 mmol) of ethyl 4-nitrophenyl carbonate. The reaction mixture is subsequently evaporated to dryness, the residue is taken up in 20 ml of methylene chloride, and the methylene chloride solution is washed three times with 20 ml of water each time. After the organic phase has been dried over sodium sulfate and the solvent has been stripped off, the crude product is purified by column chromatography on silica gel (methylene chloride/methanol: 99/1), giving 200 mg of the title compound as an amorphous powder, MALDI-MS 580.

Reaction of N-(3-amidinobenzyl)-N-isobutyl-N′-(2′-methanesulfonylbiphenyl-4-yl)oxalamide (Ex.1) and a corresponding 4-nitrophenyl carbonate analogously to Example 12 gives the following carbamates:

### EXAMPLE 13

2,2,2-trichloroethyl [imino-(3-{[isobutyl-(2′-sulfamoylbiphenyl-4-ylaminooxalyl)amino]methyl}phenyl)methyl]carbamate, MALDI-MS 684.

### EXAMPLE 14

Allyl [imino-(3-{[isobutyl-(2′-sulfamoylbiphenyl-4-ylaminooxalyl)amino]methyl}phenyl)methyl]carbamate, MALDI-MS 592.

### EXAMPLE 15

Isopropyl [imino-(3- {[isobutyl-(2′-sulfamoylbiphenyl-4-ylaminooxalyl)amino]methyl}phenyl)methyl ]carbamate, MALDI-MS 594.

### EXAMPLE 16

Butyl [imino-(3-{[isobutyl-(2′-sulfamoylbiphenyl-4-ylaminooxalyl)amino]methyl}phenyl)methyl]carbamate, MALDI-MS 608.

### EXAMPLE 17

Isobutyl [imino-(3-{[isobutyl-(2′-sulfamoylbiphenyl-4-ylaminooxalyl)amino]methyl}phenyl)methyl]carbamate, MALDI-MS 608.

### EXAMPLE 18/1

### Ethyl 3-{[isobutyl-(2′-sulfamoylbiphenyl-4-ylaminooxalyl)amino]methyl}benzimidinate

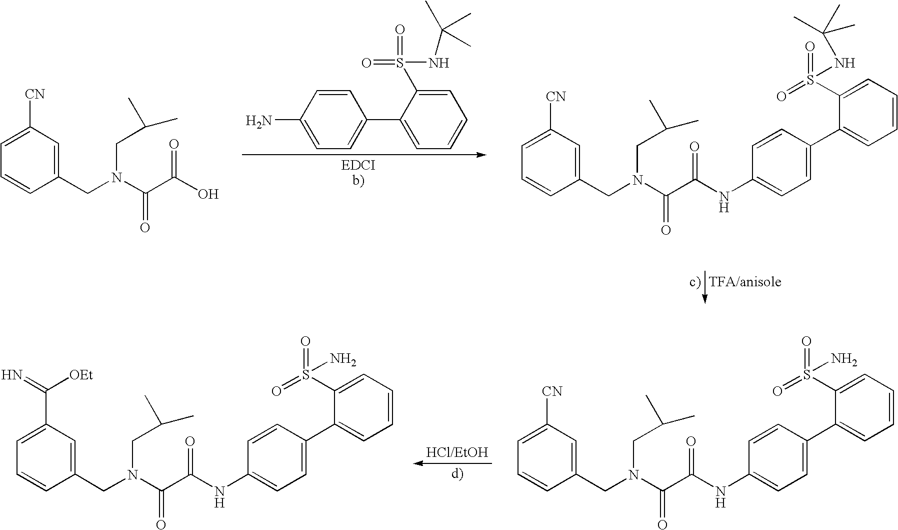

18/1a) Reaction analogously to Example 1a)-1d) gives N-(3-cyanobenzyl)-N-isobutyloxalamic acid as a yellowish oil, ESI 261.

18/1b) Reaction of the acid 18/1a) and N-tert-butyl-4′-aminobiphenyl-2-sulfonamide analogously to Example 2c) gives the compound N-(2′-tert-butylsulfamoylbiphenyl-4-yl)-N′-(3-cyanobenzyl)-N′-isobutyloxalamide as a yellow oil, ESI 547.

18/1c) Reaction of the tert-sulfonamide 18/1b) analogously to Example 1h) gives the compound N-(3-cyanobenzyl)-N-isobutyl-N′-(2′-sulfamoylbiphenyl-4-yl)oxalamide as white crystals, ESI 491.

18/1d) HCl gas is passed at −10° C. into the solution of 0.9 g (1.65 mmol) of the nitrile 18/1c) until the solution is saturated (1.5 hours). The solution is subsequently stirred at room temperature for 12 hours, the solvent is then removed by evaporation, and the residue is triturated with diethyl ether, giving 0.94 g of the title compound as the HCl salt, ESI 573.

### EXAMPLE 18

### N-[3-(N-Ethoxyamidino)benzyl]-N-isobutyl-N′-(2′-sulfamoylbiphenyl-4-yl)oxalamide

A sodium ethoxide solution prepared from 45 mg of sodium and 5 ml of ethanol, and 92 mg (0.94 mmol) of O-ethylhydroxylamine hydrochloride are added successively to the solution of 0.45 g (0.8 mmol) of 18/1d) in 5 ml of ethanol, and the mixture is refluxed for 2 hours. The ethanol is subsequently stripped off under reduced pressure, the residue is taken up in 10 ml of saturated sodium hydrogencarbonate solution, and this solution is extracted three times with 10 ml of methylene chloride each time. After the combined organic phases have been dried over sodium sulfate and the solvent has been stripped off, the crude product is purified by column chromatography on silica gel (methylene chloride/methanol: 97/3), giving 210 mg of the title compound as a white solid, ESI 552.

### EXAMPLE 19

### N-[3-(N-Methoxyamidino)benzyl]-N-isobutyl-N′-(2′-sulfamoylbiphenyl-4-yl)oxalamide

Reaction of 18/1d) and O-methylhydroxylamine analogously to Example 18 gives the title compound as a white solid, ESI 538.

### EXAMPLE 20

### N-(3-amidinobenzyl)-N′-(3-fluoro-2′-methanesulfonylbiphenyl-4-yl)-N-isobutyloxalamide

20a) Reaction of the acid 18 /1a) and 3-fluoro-2′-methanesulfonylbiphenyl-4-ylamine analogously to Example 2c) gives the compound N-(3-cyanobenzyl)-N′-(3-fluoro-2′-methanesulfonylbiphenyl-4-yl)-N-isobutyloxalamide as a colourless oil, ESI 508.

20b) Reaction of the nitrile 20a) and HCl gas in ethanol analogously to Example 18/1d) gives ethyl 3-{[(3-fluoro-2′-methanesulfonylbiphenyl-4-ylaminooxalyl)isobutylamino]methyl}benzimidinate HCl salt as colourless crystals, MALDI-MS 554.

20c) The solution of 150 mg (0.25 mmol) of compound 20b) and 160 mg of ammonium acetate in 5 ml of ethanol is refluxed for 15 hours. The reaction solution is subsequently evaporated to dryness, the residue is taken up in 10 ml of 5% sodium hydrogencarbonate solution, and the solution is extracted three times with 10 ml of methylene chloride each time. After the combined extracts have been dried over sodium sulfate and the solvent has been stripped off, the residue is recrystallised from diethyl ether, giving 50 mg of the title compound as beige crystals, MALDI-MS 525.

### EXAMPLE 21

### N-(3-Aminomethylbenzyl)-N′-(3-fluoro-2′-methanesulfonylbiphenyl-4-yl)-N-isobutyloxalamide

Hydrogenation of the nitrile 20a) with Raney nickel analogously to Example 1g) gives the title compound as pale-green crystals, MALDI-MS 512.

### EXAMPLE 22

### N-[3-(N-Ethoxyamidino)benzyl]-N′(3fluoro-2′-methanesulfonylbiphenyl-4-yl)-N-isobutyloxalamide

Reaction of 20b) and O-ethylhydroxylamine analogously to Example 18 gives the title compound as a white solid, MALDI-MS 569.

### EXAMPLE 23

### N-(3-Aminomethylbenzyl)-N-isobutyl-N′-(2′-sulfamoylbiphenyl-4-yl)oxalamide

23a) Hydrogenation of the nitrile 18/1b) analogously to Example 21 gives the compound N-(3-aminomethylbenzyl)-N′-(2′-tert-butylsulfamoylbiphenyl-4-yl)-N-isobutyloxalamide as pale-green crystals, MALDI-MS 551.

23b) Reaction of 23a) analogously to Example 1h) gives the title compound as the bistrifluoroacetate salt, MALDI-MS 495.

### EXAMPLE 24

### N-[3-(N-Hydroxyamidino)benzyl]-N-isobutyl-N′-(2′-methanesulfonylbiphenyl-4-yl)oxalamide

24a) Reaction of the acid 18/1a) and 2′-methanesulfonylbiphenyl-4-yl-amine analogously to Example 2c) gives the compound N-(3-cyano-benzyl)-N′-(2′-methanesulfonylbiphenyl-4-yl)-N-isobutyloxalamide as a colourless oil, ESI 490.

24b) A solution of 0.28 g (4 mmol) of hydroxylammonium chloride and 0.34 g (4 mmol) of sodium hydrogencarbonate in 3 ml of water was added to the solution of 0.66 g (1.34 mmol) of the nitrile 24a) in 30 ml of ethanol. The reaction mixture was subsequently refluxed for four hours and then evaporated under reduced pressure. The residue was taken up in 10 ml of water, the aqueous solution was extracted three times with 10 ml of methylene chloride each time, and the combined organic extracts were dried over sodium sulfate. After the solvent had been stripped off, the residue was recrystallised from diethyl ether, giving 0.61 g of the title compound as white crystals, MALDI-MS 523.

### EXAMPLE 25

### N-(3-Fluoro-2′-methanesulfonylbiphenyl-4-yl)-N′-[3-(N-hyd roxyamidino)benzyl]-N′-isobutyloxalamide

Reaction of the nitrile 20a) analogously to Example 24b) gives the title compound as slightly beige crystals, MALDI-MS 541.

### EXAMPLE 26

### N-[3-(N-Hydroxyamidino)benzyl]-N-isobutyl-N′-(2′-sulfamoylbiphenyl-4-yl)oxalamide

Reaction of the nitrile 18/1 c) analogously to Example 24b) gives the title compound as white crystals, MALDI-MS 524.

### EXAMPLE 27

### N-[4-(1,2-Diaminoethyl)phenyl-N′-(3-fluoro-2′-methanesulfonylbiphenyl-4-yl)oxalamide

27a) Reaction of monomethyl oxalate chloride and 3-fluoro-2′-methanesulfonylbiphenyl-4-ylamine analogously to Example 2a)-2b) gives the acid N-(3-fluoro-2′-methanesulfonylbiphenyl-4-yl)oxalamic acid as colourless crystals, FAB 338.

27b) 4 g of sodium carbonate and 4.2 g of di-tert-butyl dicarbonate are added to the solution of 2.1 g (8.26 mmol) of 1-(4-nitrophenyl)ethane-1,2-diamine (Altman, J. et. al. J. Chem. Soc. Perkin Trans.1; 1983; 365-368) in 60 ml of methanol and 30 ml of water, and the mixture is stirred at room temperature for 16 hours. The precipitate formed in the process is separated off and dissolved in 20 ml of methylene chloride. After the methylene chloride solution has been dried over sodium sulfate and the solvent has been stripped off, the solid residue is triturated with petroleum ether and filtered off with suction, giving 2.1 g of tert-butyl [2-terf-butoxycarbonylamino-2-(4-nitrophenyl)ethyl]carbamate, MALDI-MS (M+Na)⁺: 404.

27c) The mixture of 2 g (5.24 mmol) of the nitro compound 27b) and 1 g of 5% palladium/carbon in 20 ml of methanol is hydrogenated at atmospheric pressure for 15 hours. The catalyst is subsequently filtered off, and the solvent is stripped off under reduced pressure, giving 1.7 g of tert-butyl [2-(4-aminophenyl)-2-tert-butoxycarbonylaminoethyl]carbamate as a colourless oil, MALDI-MS (M+Na)⁺374.

27d) Reaction of the acid 27a) and the aniline 27c) analogously to Example 2c) gives the compound tert-butyl (2-tert-butoxycarbonylamino-2-{4-[(3-fluoro-2′-methanesulfonylbiphenyl-4-ylaminooxalyl)amino]phenyl}-ethyl)carbamate, ESI 671.

27e) Reaction of 27d) analogously to Example 1h) gives the title compound as the bistrifluoroacetate salt in the form of pale-beige crystals, ESI 471.

### EXAMPLE 28

### N-[4-(1,2-Diaminoethyl)phenyl]-N′-(2′-sulfamoylbiphenyl-4-yl)oxalamide

28a) Reaction of the acid 2b) and the aniline 27c) analogously to Example 2c) gives the compound tert-butyl (2-tert-butoxycarbonylamino-2-{4-[(2′-tert-butylsulfamoylbiphenyl-4-ylaminooxalyl)amino]phenyl}ethyl)carbamate, ESI 710.

28b) Reaction of 28a) analogously to Example 1h) gives the title compound as the bistrifluoroacetate salt, ESI 454.

### EXAMPLE 29

Reaction analogously to Example 27 gives N-[4-(1,2-diaminoethyl)phenyl]-N′-(2′-methanesulfonylbiphenyl-4-yl)oxalamide, ESI 453.

### EXAMPLE 30

### N-(3-amidinobenzyl)-N′-[3-(methanesulfonylaminomethyl)phenyl]-N-(2,2,2-trifluoroethyl)oxalamide

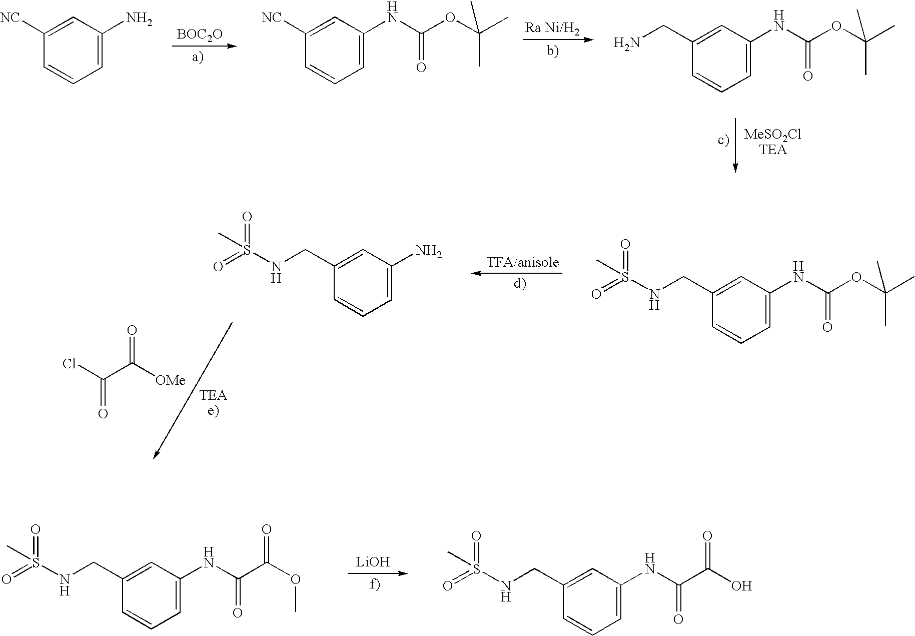

30a) 48 g of di-tert-butyl dicarbonate are added to a solution of 23.6 g (0.2 mol) of 3-aminobenzonitrile and 20.2 g of N-methylmorpholine in 500 ml of dioxane, and the mixture is stirred at 40° C. for 18 hours. The solvent is subsequently stripped off under reduced pressure, the residue is dissolved in 100 ml of methylene chloride, and the methylene chloride solution is washed three times with 50 ml of water each time. After the organic phase has been dried over sodium sulfate and the solvent has been stripped off, the crude product is purified by column chromatography on silica gel (methylene chloride), giving 16.8 g of tert-butyl (3-cyanophenyl)carbamate as colourless crystals, EI (M⁺) 218.

30b) Hydrogenation of the nitrile 30a) with Raney nickel analogously to Example 1g) gives tert-butyl (3-aminomethylphenyl)carbamate as a white powder, ESI 223.

30c) 5.1 g of methanesulfonyl chloride are added dropwise to the solution of 8.8 g (40 mmol) of the benzylamine 30b) and 5.0 g of triethylamine in 100 ml of CH₂Cl₂, and the mixture is stirred at room temperature for 15 hours. The reaction solution is then washed three times with 30 ml of water each time, and the methylene chloride solution is dried over sodium sulfate, the solvent is stripped off under reduced pressure. The oil remaining is crystallised with ethyl acetate, giving 7 g of tert-butyl [3-(methanesulfonylaminomethyl)phenyl]carbamate, ESI (M+Na)⁺323.

30d)e)f) Reaction of 30c) analogously to Example 1h), 1c) and 1d) gives the acid N-(3-(methanesulfonylaminomethyl)phenyl]oxalamic acid, MALDI-MS 273.

30g) Reaction of the acid 30f) and thionyl chloride analogously to Example 1e) gives the acid chloride [3-(methanesulfonylaminomethyl)phenylamino]-oxoacetyl chloride, which is reacted without further purification.

30h) Reaction of tert-butyl (2,2,2-trifluoroethyl)carbamate and 3-(3-bromomethylphenyl)-5-methyl-1,2,4-oxadiazole analogously to Example 1a) and 1b) gives the amine [3-(5-methyl-1,2,4-oxadiazol-3-yl)benzyl]-(2,2,2-trifluoroethyl)amine as the hydrochloride in the form of white crystals, ESI 272.

30i) Reaction of the acid chloride 30g) and the amine 30h) analogously to Example 1f) gives the compound N-[3-(methanesulfonylaminomethyl)phenyl]-N′-[3-(5-methyl-1,2,4-oxadiazol-3-yl)benzyl]-N′-(2,2,2-trifluoroethyl)oxalamide as a white solid, ESI 526.

30j) Reaction of 30i) and Raney nickel analogously to Example 1g) gives the title compound as the acetate salt

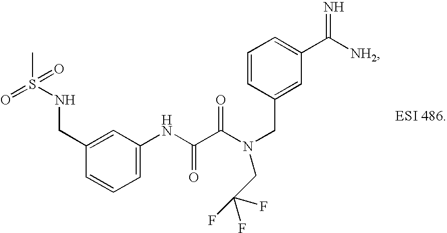

### EXAMPLE 31

### N-(4-Chlorobenzyl)-N-isobutyl-N′-[3-(methanesulfonylaminomethyl)phenyl]oxalamide

The title compound is obtained from the acid chloride 30g) and (4-chlorobenzyl)isobutylamine analogously to Example 1f), ESI 452.

### EXAMPLE 32

### N-(4-Chlorobenzyl)-N′-[3-(methanesulfonylaminomethyl)phenyl]-N-(2,2 2-trifluoroethyl)oxalamide

The title compound is obtained from the acid chloride 30g) and (4-chlorobenzyl)(2,2,2-trifluoroethyl)amine analogously to Example 1f), ESI 478.

### EXAMPLE 33

### N-(3-amidinobenzyl)-N-isobutyl-N′-[3-(methanesulfonylaminomethyl)phenyl]oxalamide

Reaction of the acid chloride 30g) and the amine 1b) analogously to Example 30i) and 30j) gives the title compound as the acetate salt, ESI 460.

### EXAMPLE 34

### N-(3-Carbamoylbenzyl)-N′-(3-fluoro-2′-methanesulfonylbiphenyl-4-yl)-N-(2,2,2-trifluoroethyl)oxalamide

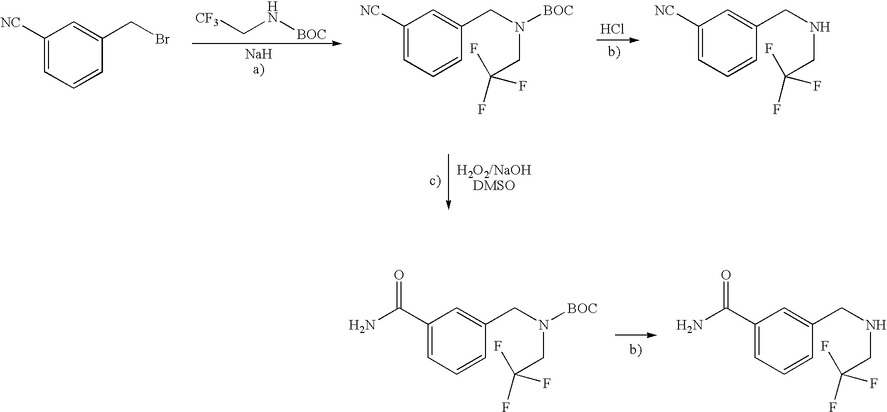

34a) Reaction of tert-butyl (2,2,2-trifluoroethyl)carbamate and 3-bromomethylbenzonitrile analogously to Example 1 a) gives the compound tertbutyl (3-cyanobenzyl)(2,2,2-trifluoroethyl)carbamate as a colourless oil, ESI 315.

34b) Reaction of 34a) analogously to Example 1b) gives the amine 3-[(2,2,2-trifluoroethylamino)methyl]benzonitrile as the HCl salt in the form of white crystals, ESI 215.

34c) 0.8 ml of dimethyl sulfoxide, 2.2 g of potassium carbonate and 1.6 ml of 30% hydrogen peroxide are added successively with ice cooling to a solution of 1 g (3.2 mmol) of the nitrile 34a) in 20 ml of methanol, and the mixture is subsequently stirred at room temperature for 12 hours. 20 ml of water are added, and the precipitate is then filtered off with suction, washed with 10 ml of water and dried in air, giving 0.95 g of tert-butyl (3-carbamoylbenzyl)(2,2,2-trifluoroethyl)carbamate as colourless crystals, ESI 333.

34d) Reaction of 34c) analogously to Example 1b) gives the amine 3-[(2,2,2-trifluoroethylamino)methyl]benzamide as the HCl salt, ESI 233.

34e) Reaction of the acid 27a) and the amine 34d) analogously to Example 2c) gives the title compound as colourless crystals,

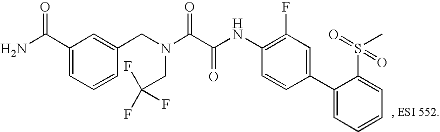

### EXAMPLE 35

### N-(3-arbamoylbenzyl)-N′-(3-fluoro-2′-methanesulfonylbiphenyl-4-yl)-N-isobutyloxalamide

35a) Reaction of the compound tert-butyl (3-cyanobenzyl)isobutylcarbamate prepared in Example 18/1a) analogously to Example 34c) gives the benzamide tert-butyl (3-carbamoylbenzyl)isobutylcarbamate as white crystals, ESI 307.

35b) Reaction of 35a) analogously to Example 1b) gives the amine 3-(isobutylaminomethyl)benzamide, ESI 207.

35c) Reaction of the acid 27a) and the amine 35b) analogously to Example 2c) gives the title compound as colourless crystals, ESI 526.

### EXAMPLE 36

### N-(3-Carbamoylbenzyl)-N-isobutyl-N′-[4-(2-oxopiperidin-1-yl)phenyl]oxalamide

36a) Reaction of the acid 18/1a) and 1-(4-aminophenyl)piperidin-2-one analogously to Example 18/1b) gives the compound N-(3-cyanobenzyl)-N-isobutyl-N′-[4-(2-oxopiperidin-1-yl)phenyl]oxalamide as colourless crystals, ESI 433.

36b) Reaction of the nitrile 36a) analogously to Example 34c) gives the title compound as colourless crystals, ESI 451.

### EXAMPLE 37

### N-(3-Carbamoylbenzyl)-N-isobutyl-N′-[4-(2-oxopyrrolidin-1-yl)phenyl]oxalamide

37a) Reaction of the acid 18/1a) and 1-(4-aminophenyl)pyrrolidin-2-one analogously to Example 18/1b) gives the compound N-(3-cyanobenzyl)-N-isobutyl-N′-[4-(2-oxopyrrolidin-1-yl)phenyl]oxalamide as grey crystals, ESI 419.

37b) Reaction of the nitrile 37a) analogously to Example 34c) gives the title compound as colourless crystals, ESI 437.

### EXAMPLE 38

Reaction of the nitrile 36a) analogously to Example 5b) gives N-(3-amidinobenzyl)-N-isobutyl-N′-[4-(2-oxopiperidin-1-yl)phenyl]oxalamide as white crystals, ESI 450.

### EXAMPLE 39

Reaction of the nitrile 37a) analogously to Example 5b) gives N-(3-amidinobenzyl)-N-isobutyl-N′-[4-(2-oxopyrrolidin-1-yl)phenyl]oxalamide as white crystals, ESI 436.

### EXAMPLE 40

Hydrogenation of the nitrile 36a) with Raney nickel analogously to Example 30b) gives N-(3-aminomethylbenzyl)-N-isobutyl-N′-[4-(2-oxopiperidin-1-yl)phenyl]oxalamide as white crystals, ESI 437.

### EXAMPLE 41

Hydrogenation of the nitrile 37a) with Raney nickel analogously to Example 30b) gives N-(3-aminomethylbenzyl)-N-isobutyl-N′-[4-(2-oxopyrrolidin-1-yl)phenyl]oxalamide as grey crystals, ESI 423.

### EXAMPLE 42

### N-(3-Amidinobenzyl)-N′-[4-(2-oxopiperidin-1-yl)phenyl]-N-(2,2,2-trifluoroethyl)oxalamide

42a) Reaction of the amine 34b) with monomethyl oxalate chloride analogously to 1c) followed by saponification of the resultant methyl ester analogously to 1 d) gives the acid N-(3-cyanobenzyl)-N-(2,2,2-trifluoroethyl)oxalamic acid as a colourless oil, ESI 287.

42b) Reaction of the acid 42a) and 1-(4-aminophenyl)piperidin-2-one analogously to Example 18/1b) gives the compound N-(3-cyanobenzyl)-N′-[4-(2-oxopiperidin-1-yl)phenyl]-N-(2,2,2-trifluoroethyl)oxalamide as white crystals, ESI 459.

42c) Reaction of the nitrile 42b) analogously to Example 5b) gives the title compound as white crystals, ESI 476.

### EXAMPLE 43

Reaction of the nitrile 42b) analogously to Example 34c) gives the compound N-(3-carbamoylbenzyl)-N′-[4-(2-oxopiperidin-1-yl)phenyl]-N-(2,2,2-trifluoroethyl)oxalamide as grey crystals, ESI 477.

### EXAMPLE 44

Hydrogenation of the nitrile 42b) with Raney nickel analogously to Example 30b) gives N-(3-aminomethylbenzyl)-N′-[4-(2-oxopiperidin-1-yl)phenyl]-N-(2,2,2-trifluoroethyl)oxalamide as white crystals, ESI 463.

### EXAMPLE 45

### N-(3-Carbamoylbenzyl)-N′-[4-(2-oxoazepan-1-yl)phenyl]-N-(2.2,2-trifluoroethyl)oxalamide

45a) Reaction of the acid 42a) and 1-(4-aminophenyl)azepan-2-one analogously to Example 18/1b) gives the compound N-(3-cyanobenzyl)-N′-[4-(2-oxoazepan-1-yl)phenyl]-N-(2,2,2-trifluoroethyl)oxalamide as white crystals, ESI 473.

45b) Reaction of the nitrile 45a) analogously to Example 34c) gives the title compound as white crystals, ESI 491.

### EXAMPLE 46

Reaction of the nitrile 45a) analogously to Example 5b) gives the compound N-(3-amidinobenzyl)-N′-[4-(2-oxoazepan-1-yl)phenyl]-N-(2,2,2-trifluoroethyl)oxalamide as white crystals, ESI 490.

### EXAMPLE 47

Hydrogenation of the nitrile 45a) with Raney nickel analogously to Example 30b) gives the compound N-(3-aminomethylbenzyl)-N′-[4-(2-oxoazepan-1-yl)phenyl]-N-(2,2,2-trifluoroethyl)oxalamide as the HCl salt, ESI 477.

### EXAMPLE 48

### N-(3-Carbamoylbenzyl)-N-isobutyl-N′-[4-(2-oxoazepan-1-yl)phenyl]oxalamide

48a) Reaction of the acid 18/1a) and 1-(4-aminophenyl)azepan-2-one analogously to Example 18/1b) gives the compound N-(3-cyanobenzyl)-N-isobutyl-N′-[4-(2-oxoazepan-1-yl)phenyl]oxalamide as colourless crystals, ESI 447.

48b) Reaction of the nitrile 48a) analogously to Example 34c) gives the title compound as colourless crystals, ESI 465.

### EXAMPLE 49

Reaction of the nitrile 48a) analogously to Example 5b) gives the compound N-(3-amidinobenzyl)-N-isobutyl-N′-[4-(2-oxoazepan-1-yl)phenyl])-oxalamide as white crystals, ESI 464.

### EXAMPLE 50

Hydrogenation of the nitrile 48a) with Raney nickel analogously to Example 30b) gives the compound N-(3-aminomethylbenzyl)-N-isobutyl-N′-[4-(2-oxoazepan-1-yl)phenyl]oxalamide as the HCl salt, ESI 451.

### EXAMPLE 51

### N-(3-Amidinobenzyl)-N′-(2′-methanesulfonylbiphenyl-4-yl)-N-(2,2,2-trifluoroethyl)oxalamide

51 a) Reaction of the acid 42a) and 2′-methanesulfonylbiphenyl-4-ylamine analogously to Example 18/1b) gives the compound N-(3-cyanobenzyl)N′-(2′-methanesulfonylbiphenyl-4-yl)-N-(2,2,2-trifluoroethyl)oxalamide, ESI 516.

51b) Reaction of the nitrile 51a) analogously to Example 5b) gives the title compound, ESI 533.

### EXAMPLE 52

### N-(3-amidinobenzyl)-N′-(3-fluoro-2′-methanesulfonylbiphenyl-4-yl)-N-(2,2,2-trifluoroethyl)oxalamide

52a) Reaction of the acid 42a) and 3-fluoro-2′-methanesulfonylbiphenyl-4-ylamine analogously to Example 18/1b) gives the compound N-(3-cyanobenzyl)-N′-(3-fluoro-2′-methanesulfonylbiphenyl-4-yl)-N-(2,2,2-trifluoroethyl)oxalamide, ESI 534.

52b) Reaction of the nitrile 52a) analogously to Example 5b) gives the title compound, ESI 551.

### EXAMPLE 53

### N-(3-amidinobenzyl)-N′-(2′-sulfamoylbiphenyl-4-yl)-N-(2,2,2-trifluoroethyl)oxalamide

53a) Reaction of the acid 42a) and N-tert-butyl-4′-aminobiphenyl-2-sulfonamide analogously to Example 18/1b) gives the compound N-(2′-tert-butylsulfamoylbiphenyl-4-yl)-N′-(3-cyanobenzyl)-N′-(2,2,2-trifluoroethyl)oxalamide, ESI 573.

53b) Reaction of the nitrile 53a) analogously to Example 5b) gives the compound N-(2′-tert-butylsulfamoylbiphenyl-4-yl)-N′-(3-amidinobenzyl)-N′-(2,2,2-trifluoroethyl)oxalamide, ESI 590.

53c) Reaction of the compound 53b) analogously to Example 1h) gives the title compound, ESI 534.

The examples below relate to pharmaceutical preparations:

### EXAMPLE A

### Injection Vials

A solution of 100 g of an active ingredient of the formula I and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### EXAMPLE B

### Suppositories

A mixture of 20 g of an active ingredient of the formula I is melted with 100 g of soya lecithin and 1400 g of cocoa butter, poured into moulds and allowed to cool. Each suppository contains 20 mg of active ingredient.

### EXAMPLE

### Solution

A solution is prepared from 1 g of an active ingredient of the formula I, 9.38 g of NaH₂PO₄.2 H₂O, 28.48 g of Na₂HPO₄.12 H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

### Ointment

500 mg of an active ingredient of the formula I are mixed with 99.5 g of Vaseline under aseptic conditions.

### EXAMPLE E

### Tablets

A mixture of 1 kg of active ingredient of the formula I, 4 kg of lactose, 1.2 kg of potato starch, 0.2 kg of talc and 0.1 kg of magnesium stearate is pressed in a conventional manner to give tablets in such a way that each tablet contains 10 mg of active ingredient.

### EXAMPLE F

### Coated Tablets

Tablets are pressed analogously to Example E and subsequently coated in a conventional manner with a coating of sucrose, potato starch, talc, tragacanth and dye.

### EXAMPLE G

### Capsules

2 kg of active ingredient of the formula I are introduced in a conventional manner into hard gelatine capsules in such a way that each capsule contains 20 mg of the active ingredient.

### EXAMPLE H

### Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

1. Compounds of the formula I 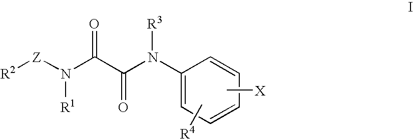
in which
R¹ and R³, independently of one another, are H or A, Ar, Ar-alk, Het, Het-alk or acyl,
R² is Ar or Het,
R⁴ is H, A, OH, OA′, OAr, Ar-alk-O, O-acyl, COOH, COON, CONH₂, CONHA′, CONA′2, CN, NHA′, NA′2, NHCH2Ar′, NH-acyl or Hal,
X is Ar, Ar-alk or U,
Ar is phenyl, naphthyl or biphenyl, each of which is unsubstituted or monosubstituted or polysubstituted by A′, Hal, OH, OA′, OCH2Ar′, O-acyl, COOH, COON, CONH₂, CONHA′, CONA′₂, CONHNH₂, CH2NH2, CH2NHA′, CH₂NA′₂, CH2CH2NH2, CH2NH-acyl, CN, NH2, NHA′, NA′2, NHCH₂Ar′, NHCOAr′, C(═NH)NH2, C(═NH)NH—COOA′, SO2CH2R⁶,
SO₂NR⁸R⁹, 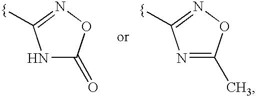
Ar′ is phenyl, naphthyl or biphenyl, each of which is unsubstituted or monosubstituted or polysubstituted by A′, Hal, OH, OA′, O-acyl, COOH, COON, CONH2, CONHA′, CONA′₂, CONHNH₂, CH2NH2, CH2NHA′, CH2NA′2, CH2CH2NH₂,
CH2NH-acyl, CN, NHA′, NA′2, C(═NH)NH₂, SO2CH2R⁶ or SO2NR⁸R⁹,
Het is a monocyclic or bicyclic aromatic heterocyclic radical having from I to 4 N, 0 and/or S atoms which is unsubstituted or monosubstituted or polysubstituted by A′, Hal, OH, OA′, OCH2Ar′, O-acyl, COOH, COON, CONH2, CONHA′, CONA′2, CONHNH2, CH2NH2, CH2NHA′, CH2NA′2, CH2CH2NH2, CH2NH-acyl, CN, NHA′, NA′₂, NHCH2Ar′, NHCOAr′, C(═NH)NH2, S02CH2R⁶, S02NR⁸R⁹, 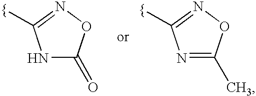
U is a radical of the formula IIa, IIb, IIc or IId 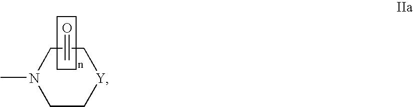
(CH₂)ₚ—SO₂—(CH₂)ₙ—R⁶ IIb,
(CH₂)ₚ—SO₂—NR⁸R⁹ IIc,
(CH₂)ₚ—NH—SO₂—(CH₂)ₙ—R⁶ IId,
which is unsubstituted or monosubstituted or polysubstituted by A′, Hal, OH, OA′, OCH2Ar′, O-acyl, COOH, COON, CONH2, CONHA′, CONA′2, CONHNH2, CH2NH₂, CH2NHA′, CH2NA′2, _{CH2CH2NH2,} CH2NH-acyl, CN, NHA′, NA′2, NHCH2Ar′, NHCOAr′, C(═NH)NH2, SO2CH2R⁶, S02NR⁸R⁹, 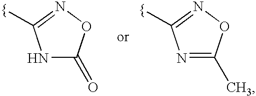
Y is 0, S, NW or an alkylene chain (CH2)ₘ, which is unsubstituted or monosubstituted or polysubstituted by OH, OA′, OAr′, O-acyl, COOH, COON, CONH2, CONHA′, _{CONA′2,} CN, NH₂, NHA′, NA′2, NHCH2Ar′, NH-acyl, NHCOAr′, C(═NH)NH2 or Hal and which may be interrupted by 0, S or NR⁵,
Z is 0, NR⁵ or an alkylene chain (CH2)ₘ, which is unsubstituted or monosubstituted or polysubstituted by OH, OA′, OAr′, O-acyl, COOH, COON, CONH2, CONHA′, CONA′₂, CN, NH2, NHA′, _{NA′2,} NHCH2Ar′, NH-acyl, NHCOAr′, C(═NH)NH2 or Hal,
A is unbranched or branched alkyl having 1-8 carbon atoms, which is unsubstituted or monosubstituted or polysubstituted by OH, OA′, OAr′, O-acyl, COOH, COON, CONH2, CONHA′, CONA′2, CN, NH2, NHA′, NA′2, NHCH2Ar′, NH-acyl, NHCOAr′, C(═NH)NH2 or Hal and in which one or two CH₂ groups may be replaced by 0 or S atoms and/or by —CH═CH— groups and/or, in addition, 1-7 H atoms may be replaced by F,
A′ is unbranched or branched alkyl having 1-8 carbon atoms,
R⁵ is H, A, Ar, Ar-alk, Het, CO-T-R⁶ or S02-T-R⁶, and, if Y=NR⁵, R⁵ may alternatively be —C(═NH)—R⁷,
T is absent or is an alkylene chain having 1-5 carbon atoms, alkenylene chain having 2-5 carbon atoms or alkynylene chain having 2-5 carbon atoms, each of which is unsubstituted or monosubstituted or polysubstituted by OH, OA′, OAr′, O-acyl, COOH, COON, CONH2, CONHA′, CONA′2, CN, NH2, NHA′, NA′2, NHCH2Ar′, NH-acyl, NHCOAr′, C(═NH)NH2 or Hal,
R⁶ is H, A, Ar, Ar-alk or Het,
R⁷ is H, A′, Ar-alk or NR^{B}R⁹,
R⁸ and R⁹, independently of one another, are H, A, Ar, Ar-alk, Het, acyl, Q¹ or Q², or, together with the nitrogen to which they are bonded, are a monocyclic saturated, unsaturated or aromatic heterocyclic radical having from 1 to 3 N, 0 and/or S atoms, which is unsubstituted or monosubstituted or polysubstituted by A′, Hal, OH, OA′, OCH2Ar′, O-acyl, COOH, COON, CONH2, CONHA′, CONA′2, CONHNH2, _{CH2NH2,} CH2NHA′, CH₂NA′₂, CH₂CH2NH2, CH2NH-acyl, CN, NHA′, NA′₂, NHCH2Ar′, NHCOAr′, C(═NH)NH2 or S02CH2R⁶,
Q¹ is a cycloalkyl radical which is unsubstituted or monosubstituted or disubstituted by A′,
Q² is a monocyclic saturated or unsaturated heterocyclic radical having from 1 to 3 N, 0 and/or S atoms which is unsubstituted or monosubstituted or disubstituted by A′, Hal, OH, OA′, OCH2Ar′, O-acyl, COOH, COON, CONH2, CONHA′, CONA′2, CONHNH2, _{CH2NH2,} CH2NHA′, CH2NA′2, CH2CH2NH2, CH2NH-acyl, CN, NHA′, NA′₂, NHCH2Ar′, NHCOAr′, C(═NH)NH2 or S02CH2R⁶,
Hal is F, Cl, Br or I,
alk is alkylene having 1, 2, 3, 4, 5 or 6 carbon atoms,
m is 0, 1, 2, 3 or 4,
n is 1, 2or 3,
p is 1, 2, 3, 4 or 5,
and pharmaceutically tolerated salts, solvates and stereoisomers thereof.

2. Compounds according to claim 1, in which
R¹is H, A or Ar-alk,
and pharmaceutically tolerated salts, solvates and stereoisomers thereof.

3. Compounds according to claim 1, in which
R² is Ar,
and pharmaceutically tolerated salts, solvates and stereoisomers thereof.

4. Compounds according to claim 1, in which
R¹ is H, alkyl having 1, 2, 3, 4, 5 or 6 carbon atoms, in which 1-5 H atoms may be replaced by F, or benzyl,
and pharmaceutically tolerated salts, solvates and stereoisomers thereof.

5. Compounds according to claim 1, in which
R² is phenyl which is monosubstituted or disubstituted by Hal, OH, OA′, COOH, COON, CONH2, CONHNH2, CH2NH2, CH2NHA′, CH(NH2)CH2NH2, C(═NH)NH2, C(═NH)NH—COOA′,
SO2NR⁸R⁹, or 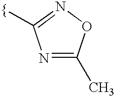
and pharmaceutically tolerated salts, solvates and stereoisomers thereof.

6. Compounds according to claim 1, in which Z is an unsubstituted alkylene chain (CH2)ₘ and
m is 0 or 1,
and pharmaceutically tolerated salts, solvates and stereoisomers thereof.

7. Compounds according to claim 1, in which
R³ is H or A,
and pharmaceutically tolerated salts, solvates and stereoisomers thereof.

8. Compounds according to claim 1, in which
R⁴ is H, F or CI,
and pharmaceutically tolerated salts, solvates and stereoisomers thereof.

9. Compounds according to claim 1, in which
X is phenyl which is monosubstituted or disubstituted by CH2CH2NH2, C(═NH)NH2, SO2CH2R⁶ or SO2NR⁸R⁹,
or an unsubstituted radical of the formula IIa, IIb, IIc or IId 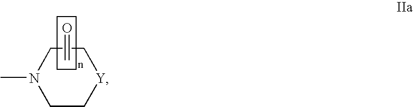
(CH₂)ₚ—SO₂—(CH₂)ₙ—R⁶ lIb,
(CH₂)ₚ—SO₂—NR⁸R⁹ IIc,
(CH₂)ₚ—NH—SO₂—(CH₂)ₙ—R⁶ IId,
and pharmaceutically tolerated salts, solvates and stereoisomers thereof.

10. Compounds according to claim 1, in which
X is phenyl which is monosubstituted or disubstituted by CH₂CH2NH2, C(═NH)NH₂, SO2CH2R⁶ or SO2NR⁸R⁹, or an unsubstituted radical of the formula IIa or IId 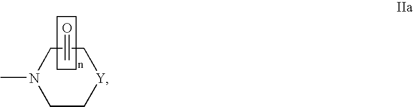
(CH₂)ₚ—NH—SO₂—(CH₂)ₙ—R⁶ IId,
and pharmaceutically tolerated salts, solvates and stereoisomers thereof.

11. Compounds according to claim 1, in which
X is phenyl which is monosubstituted or disubstituted by CH2CH2NH2, C(═NH)NH2, SO2A″ or SO2NH₂, or an unsubstituted radical of the formula IIa or lid 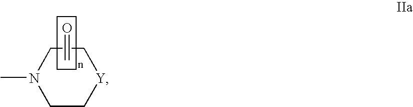
(CH₂)ₚ—NH—SO₂—CH₃ IId,
A″ is alkyl having 1, 2, 3, 4, 5 or 6 carbon atoms,
n is 1 or 2,
p is 1 or 2,
Y is 0, NR⁵ or an unsubstituted alkylene chain (CH2)m′,
m′ is 0, 1 or 2,
and pharmaceutically tolerated salts, solvates and stereoisomers thereof.

12. Compounds according to claim 1, in which
R⁵ is H,
and pharmaceutically tolerated salts, solvates and stereoisomers thereof.

13. Compounds according to claim 1, in which
T is absent,
and pharmaceutically tolerated salts, solvates and stereoisomers thereof.

14. Compounds according to claim 1, in which
R⁶ is H or A,
and pharmaceutically tolerated salts, solvates and stereoisomers thereof.

15. Compounds according to claim 1, in which
R⁷ is NH2,
and pharmaceutically tolerated salts, solvates and stereoisomers thereof.

16. Compounds according to claim 1, in which
R⁸ is H,
and pharmaceutically tolerated salts, solvates and stereoisomers thereof.

17. Compounds according to claim 1, in which
R⁹ is H, A, benzyl, Het, Q¹ or Q²,
and pharmaceutically tolerated salts, solvates and stereoisomers thereof.

18. Compounds according to claim 1, in which
R⁸ and R⁹, together with the nitrogen to which they are bonded, are pyrrolidinyl, piperidinyl, piperazinyl, morpholinyl, tetrahydropyrimidinyl, dihydropyridinyl or dihydroimidazolyl, and pharmaceutically tolerated salts, solvates and stereoisomers thereof.

19. Compounds according to claim 1, in which
Ar is phenyl which is monosubstituted by Hal, OH, OA′, COOH, COON, CONH2, CONHNH2, CH2NH2, CH2CH2NH2, CH₂NHA′, CH(NH2)CH2NH2, C(═NH)NH2, C(═NH)NH—COOA′, SO2A′, S02NR⁸R⁹, or 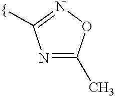
and pharmaceutically tolerated salts, solvates and stereoisomers thereof.

20. Compounds according to claim 1, in which
Ar′ is phenyl,
and pharmaceutically tolerated salts, solvates and stereoisomers thereof.

21. Compounds according to claim 1, in which
U is an unsubstituted radical of the formula IIa or IId, 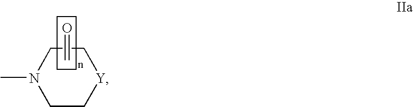
(CH₂)ₚ—NH—SO₂—(CH₂)ₙ—R⁶ IId,
and pharmaceutically tolerated salts, solvates and stereoisomers thereof.

22. Compounds according to claim 1, in which
U is an unsubstituted radical of the formula IIa or IId 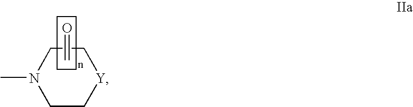
(CH₂)ₚ—NH—SO₂—CH₃ IId,
n is 1 or 2,
p is 1 or 2,
Y is 0, NR⁵ or an unsubstituted alkylene chain (CH2)m, R⁵ is H,
m is 0, 1 or 2,
and pharmaceutically tolerated salts, solvates and stereoisomers thereof.

23. Compounds according to claim 1, in which
Q² is pyrrolidinyl, piperidinyl, piperazinyl, morpholinyl, tetrahydropyrimidinyl, dihydropyridinyl or dihydroimidazolyl,
and pharmaceutically tolerated salts, solvates and stereoisomers thereof.

24. Compounds according to claim 1, in which
the radical of the formula IIa is:
morpholin4-yl, 2-oxopiperidin-1-yl, 2-oxopyrrolidin-1-yl, 2oxo-1H-pyridin-1-yl, 3-oxomorpholin-4-yl, 4-oxo-1 H-pyridin-1yl, 2,6-dioxopiperidin-1-yi, 2-oxopiperazin-1-yl, 2,5-dioxopyrrolidin-1-yl, 2-oxo-1,3-oxazolidin-3-yl, 3-oxo-2H-pyridazin2-yl or 2-caprolactam-1-yl,
and pharmaceutically tolerated salts, solvates and stereoisomers thereof.

25. Compounds according to claim 1, in which and pharmaceutically tolerated salts, solvates and stereoisomers thereof.
Rⁱ is H, alkyl having 1, 2, 3, 4, 5 or 6 carbon atoms, in which 1-5 H atoms may be replaced by F, or benzyl,
R² is phenyl which is monosubstituted or disubstituted by Hal, OH, OA′, COOH, COON, CONH₂ᵢ CONHNH2, CH2NH2, CH2NHA′, CH(NH2)CH2NH₂, C(═NH)NH2, C(═NH)NH—COOA′, S02NR⁸R⁹, or 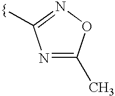
Z is an unsubstituted alkylene chain (CH2)ₘ, m is 0 or 1,
R³ is H or A,
R⁴ is H, F or Cl,
X is phenyl which is monosubstituted or disubstituted by CH2CH2NH2, C(═NH)NH2, SO2A″ or S02NH2, or an unsubstituted radical of the formula IIa or lid 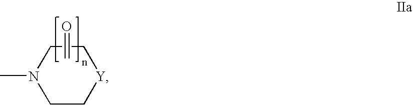
(CH₂)ₚ—NH—SO₂—CH₃ IId,
A″ is alkyl having 1, 2, 3, 4, 5 or 6 carbon atoms,
n is 1 or 2,
p is 1 or 2,
Y is 0, NR⁵ or an unsubstituted alkylene chain (CH2)m′,
m′ is 0, 1 or 2,
and pharmaceutically tolerated salts, solvates and sfereoisomers thereof.

26. Compounds according to claim 1, selected from the group consisting of
N-(3-amidinobenzyl)-N-isobutyl-M-(2′-sulfamoylbiphenyl-4-yl)oxalamide,
N-(2′-tert-butylsulfamoylbiphenyl-4-yl)-N′-(3-amidinobenzyl)-W-iso-butyloxalamide,
N-(3-amidinobenzyl)-W-(2′-sulfamoylbiphenyl-4-yl)oxalamide, N-(3-amidinobenzyl)-N-isobutyl-N′-(2′-methanesulfonylbiphenyl-4-yl)oxalamide, methyl [imino-(3-{[isobutyl-(2′-methanesulfonylbiphenyl-4-ylaminooxalyl)amino]methyl}phenyl)methyl]carbamate, N-(3-amidinobenzyl)-W-(4′-amidinobiphenyl-4-yl)-N-isobutyloxalamide,
N-(4′-aminomethylbiphenyl-4-yl)-M-(3-amidinobenzyl)-M-isobutyloxalamide, 3-{[isobutyl-(2′-sulfamoylbiphenyl-4-ylaminooxalyl)amino]methyl}benzoic acid,
N-(3-amidinophenyl)-N-isobutyl-W-(2′-sulfamoylbiphenyl-4-yl)oxalamide,
N-(3-amidinophenyl)-N-isobutyl-M-(2′-sulfamoylbiphenyl-4-yl)oxalamide,
N-(3-hydrazinocarbonylbenzyl)-N-isobutyl-M-(2′-sulfamoylbiphenyl-4-yl)oxalamide,
N-benzyl-N-(3-amidinobenzyl)-M-(2′-sulfamoylbiphenyl-4-yl)oxalamide, ethyl [imino-(3-{[isobutyl(2′-sulfamoylbiphenyl-4-ylaminooxalyl)amino]methyl}phenyl)methyl]carbamate, 2,2,2-trichloroethyl[imino-(3-{[isobutyl-(2′-sulfamoylbiphenyl-4-ylaminooxalyl)amino]methyl}phenyl)methyl]carbamate, allyl [imino-(3-{[isobutyl-(2′-sulfamoylbiphenyl-4-ylarminooxalyl)amino]methyl}phenyl)methyl]carbamate, isopropyl [imino-(3-{[isobutyl-(2′-sulfamoylbiphenyl-4-ylaminooxalyl)amino]methyl)phenyl)methyl]carbamate, butyl [imino-(3-{[isobutyl-(2′-sulfamoylbiphenyl-4-ylaminooxalyl)amino]methyl}phenyl)methyl]carbamate, isobutyl [imino-(3-{[isobutyl-(2′-sulfamoylbiphenyl-4-ylaminooxalyi)amino]methyl}phenyl)methyl]carbamate, ethyl 3-{[isobutyl-(2′-sulfamoylbiphenyl-4-ylaminooxalyl)amino]methyl}benzimidate,
N-[3-(N-ethoxyamidino)benzyl]-N-isobutyl-N-(2′-sulfamoylbiphenyl-4-yl)oxalamide,
N-[3-(N-methoxyamidino)benzyl]-N-isobutyl-N-(2′-sulfamoylbiphenyl-4-yl)oxalamide,
N-(3-amidinobenzyl)-N-(3-fluoro-2′-methanesulfonylbiphenyl-4-yl)-N-isobutyloxalamide,
N-(3-aminomethylbenzyl)-N-(3-fluoro-2′-methanesulfonylbiphenyl-4yl)-N-isobutyloxalamide,
N-[3-(N-ethoxyamidino)benzyl]-N-(3-fluoro-2′-methanesulfonylbiphenyl-4-yi)-N-isobutyloxalamide, N-(3-aminomethylbenzyl)-N-isobutyl-N-(2′-sulfamoylbiphenyl-4-yl)oxalamide,
N-[3-(N-hydroxyamidino)benzyl]-N-isobutyl-N-(2′-methanesulfonylbiphenyl-4-yl)oxalamide,
N-(3-fluoro-2′-methanesulfonylbiphenyl-4-yi)-N-[3-(N-hydroxyamidino)benzyl]-N-isobutyloxalamide, N-[3-(N-hydroxyamidino)benzyl]-N-isobutyl-N-(2′-sulfamoylbiphenyl4-yl)oxalamide,
N-[4-(1,2-diaminoethyl)phenyl]-N-(3-fluoro-2′-methanesulfonylbiphenyl-4-yl)oxalamide,
N-[4-(1 ,2-diaminoethyl)phenyl]-W-(2′-sulfamoylbiphenyl-4-yl)oxalamide,
N-(3-amidinobenzyl)-M-[3-(methanesulfonylaminomethyl)phenyl]-N(2,2,2-trifluoroethyl)oxalamide,
N-(4-chlorobenzyl)-N-isobutyl-W-[3-(methanesulfonylaminomethyl)-phenyl]oxalamide,
N-(4-chlorobenzyl)-W-[3-(methanesulfonylaminomethyl) phenyl]-N(2,2,2-trifluoroethyi)oxalamide,
N-(3-amidinobenzyl)-N-isobutyl-W-[3-(methanesulfonylaminomethyl)phenyl]oxalamide,
N-(3-carbamoylbenzyl)-M-(3-fluoro-2′-methanesulfonylbiphenyl-4-yi)N-(2,2,2-trifluoroethyl)oxalamide,
N-(3-carbamoylbenzyl)-M-(3-fluoro-2′-methanesulfonylbiphenyl-4-yl)N-isobutyloxalamide,
N-(3-amidinobenzyl)-N-isobutyl-M-[4-(2-oxopiperidin-1-yl)phenyl]oxalamide,
N-(3-amidinobenzyl)-N-isobutyl-W-[4-(2-oxopyrrolidin-1-yI)phenyl]-oxalamide,
N-(3-aminomethylbenzyl)-N-isobutyl-M-[4-(2-oxopiperidin-1-yl)-phenyl]oxalamide,
N-(3-aminomethylbenzyl)-N-isobutyl-M-[4-(2-oxopyrrolidin-1-yl)phenyl]oxalamide,
N-(3-am idinobenzyl)-W-[4-(2-oxopiperidin-1-yi)phenyl]-N-(2,2,2-trifluoroethyl)oxalamide,
N-(3-carbamoylbenzyl)-W-[4-(2-oxopiperidin-1-yl)phenyl]-N-(2,2,2-trifluoroethyl)oxalamide,
N-[4-(1,2-diaminoethyl)phenyl]-W-(2′-sulfamoylbiphenyl-4-yl)oxalamide,
N-(3-amidinobenzyl)-M-[3-(methanesulfonylaminomethyl)phenyl]-N(2,2,2-trifluoroethyl)oxaIamide,
N-(4-chlorobenzyl)-N-isobutyl-W-[3-(methanesulfonylaminomethyl)phenyl]oxalamide,
N-(4-chlorobenzyl)-W-[3-(methanesulfonylaminomethyl) phenyl]-N(2,2,2-trifluoroethyi)oxalamide,
N-(3-amidinobenzyl)-N-isobutyl-W-[3-(methanesulfonylaminomethyl)phenyl]oxalamide,
N-(3-carbamoylbenzyl)-M-(3-fluoro-2′-methanesulfonylbiphenyl-4-yi)N-(2,2,2-trifluoroethyl)oxalamide,
N-(3-carbamoylbenzyl)-M-(3-fluoro-2′-methanesulfonylbiphenyl-4-yl)N-isobutyloxalamide,
N-(3-carbamoylbenzyl)-N-isobutyl-M-[4-(2-oxopiperidin-1-yl)phenyl]oxalamide,
N-(3-carbamoylbenzyl)-N-isobutyl-M-[4-(2-oxopyrrolidin-1-yl)phenyl]oxalamide,
N-(3-amidinobenzyl)-N-isobutyl-M-[4-(2-oxopiperidin-1-yl)phenyl]oxalamide,
N-(3-amidinobenzyl)-N-isobutyl-W-[4-(2-oxopyrrolidin-1-yI)phenyl]oxalamide,
N-(3-aminomethylbenzyl)-N-isobutyl-M-[4-(2-oxopiperidin-1-yl)phenyl]oxalamide,
N-(3-aminomethylbenzyl)-N-isobutyl-M-[4-(2-oxopyrrolidin-1-yl)phenyl]oxalamide,
N-(3-am idinobenzyl)-W-[4-(2-oxopiperidin-1-yi)phenyl]-N-(2,2,2-trifluoroethyl)oxalamide,
N-(3-carbamoylbenzyl)-W-[4-(2-oxopiperidin-1-yl)phenyl]-N-(2,2,2-trifluoroethyl)oxalamide,
N-(3-am inom ethyl benzyl)-M-[4-(2-oxopiperidin-1-yl)phenyl]-N-(2,2,2-trifluoroethyl)oxalamide,
N-(3-carbamoylbenzyl)-M-[4-(2-oxoazepan-1-yl)phenyl]-N-(2,2,2-trifluoroethyl)oxalamide,
N-(3-amidinobenzyl)-M-[4-(2-oxoazepan-1-yl)phenyl]-N-(2,2,2-trifluoroethyl)oxalamide,
N-(3-aminomethyl benzyl)-M-[4-(2-oxoaze pan-1-yl)phenyl]-N-(2,2,2-trifluoroethyl)oxalamide,
N-(3-carbamoylbenzyl)-N-isobutyl-M-[4-(2-oxoazepan-1-yl)phenyl]oxalamide,
N-(3-amidinobenzyl)-N-isobutyl-M-[4-(2-oxoazepan-1-yl)phenyl])oxalamide,
N-(3-aminomethylbenzyl)-N-isobutyl-M-[4-(2-oxoazepan-1-yl)phenyl]oxalamide,
N-(3-amidinobenzyl)-M-(2′-methanesulfonylbiphenyl-4-yl)-N-(2,2,2-trifluoroethyl)oxalamide,
N-(3-amidinobenzyl)-M-(3-fluoro-2′-methanesulfonylbiphenyl-4-yl)-N(2,2,2-trifluoroethyl)oxalamide,
N-(3-amidinobenzyl)-M-(2′-sulfamoylbiphenyl-4-yl)-N-(2,2,2-trifluoroethyl)oxalamide,
and pharmaceutically tolerated salts, solvates and stereoisomers thereof.

27. Process for the preparation of compounds of the formula I according to claim 1 and pharmaceutically tolerated salts and solvates thereof, characterised in that
a) they are liberated from one of their flnctional derivatives by treatment with a solvolysing or hydrogenolysing agent by
i) liberating an amidino group from the hydroxyl, oxadiazole or oxazolidinone derivative by hydrogenolysis or solvolysis,
ii) replacing a conventional amino-protecting group with hydrogen by treatment with a solvolysing or hydrogenolysing agent, or liberating an amino group protected by a conventional protecting group, or
b) a cyano group is converted into an N-hydroxyamidino group, or
c) a compound of the formula II 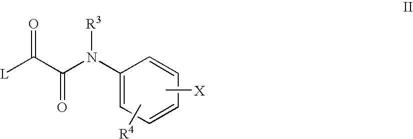
in which
L is Cl, Br, I or a free or reactively functionally modified OH group, and
R³, R⁴ and X are as defined in claim 1, with the proviso that any free amino and/or hydroxyl group present is protected,
is reacted with a compound of the formula III 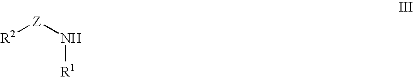
in which
R¹, R² and Z are as defined in claim 1,
and, where appropriate, a protecting group is subsequently removed or
d) a compound of the formula IV 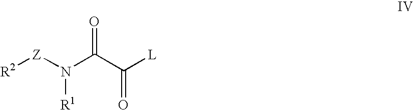
in which
L is Cl, Br, I or a free or reactively functionally modified OH group, and
R¹, R² and Z are as defined in claim 1, with the proviso that any free amino and/or hydroxyl group present is protected,
is reacted with a compound of the formula V 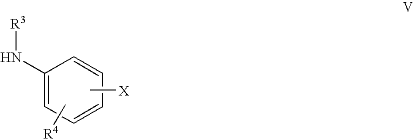
in which
R³, R⁴ and X are as defined in claim 1,
and, where appropriate, a protecting group is subsequently removed, and/or
e) a base or acid of the formula I is converted into one of its salts.

28. Compounds of the formula I according to claim 1 and physio logically acceptable salts and solvates thereof as medicaments.

29. Medicaments according to claim 28 as inhibitors of coagulation factor Xa.

30. Medicaments according to claim 28 as inhibitors of coagulation factor VIIa.

31. Medicaments according to claim 28 for the treatment of thrombosis, myocardial infarction, arteriosclerosis, inflammation, apoplexia, angina pectoris, restenosis after angioplasty, claudicatio intermittens, migraine, tumours, tumour illnesses and/or tumour metastasis.

32. Pharmaceutical preparation comprising at least one medicament according to claim 28 and, if desired, excipients and/or assistants and, if desired, other active ingredients.

33. Use of compounds according to claim 1 and/or physiologically acceptable salts and solvates thereof for the preparation of a medicament for the treatment of thrombosis, myocardial infarction, arteriosclerosis, inflammation, apoplexia, angina pectoris, restenosis after angioplasty, claudicatio intermittens, migraine, tumours, tumour illnesses and/or tumour metastasis.
